(19) United States
(12) Patent Application Publication
(10) Pub. No.: US 2024/0207405 A1
(43) Pub. Date: Jun. 27, 2024

(54) IL2 TETHERED TO ITS RECEPTOR IL2RBETA AND PORE-FORMING PROTEINS AS A PLATFORM TO ENHANCE IMMUNE CELL ACTIVITY

(71) Applicant: The General Hospital Corporation, Boston, MA (US)

(72) Inventors: Sumei Chen, Boston, MA (US); Joseph F. Cotten, Northborough, MA (US); Youssef Jounaidi, Boston, MA (US); Keith Miller, Lincoln, MA (US); Stuart Forman, Arlington, MA (US)

(21) Appl. No.: 18/288,519

(22) PCT Filed: Apr. 27, 2022
(86) PCT No.: PCT/US2022/071948

§ 371 Date: Oct. 26, 2023

Related U.S. Application Data
(60) Provisional application No. 63/181,025, filed on Apr. 28, 2021.

(51) Int. Cl. A61K 39/00; A61K 39/395; A61K 41/17; A61P 35/00; C07K 14/005; C07K 14/55; C07K 14/705; C07K 14/715; C12N 15/86

(52) U.S. Cl. CPC A61K 39/4635; A61K 39/3955; A61K 39/4611; A61K 39/4613; A61K 41/17; A61P 35/00; C07K 14/005; C07K 14/55; C07K 14/70521; C07K 14/7155; C12N 15/86; A61K 2039/54; C12N 2740/15043; C12N 2830/48

(57) ABSTRACT

Described herein are immune cells (e.g., natural killer (NK), T cells) expressing a chimeric protein comprising IL2 and IL2Rβ (e.g., CIRB), a chimeric protein comprising IL2, IL2Rβ and IL21R (e.g., CIRB21), a chimera protein comprising IL2, IL2Rβ, and CD28 (e.g., CIRB28), or a combination thereof, and comprising a nucleic acid encoding a pore-forming protein, and methods of using such immune cells for treating a subject (e.g., a subject having cancer, graft-versus-host disease (GVHD), or an autoimmune disease. Expression of the pore-forming protein can be induced to promote destruction of immune cells expressing CIRB, CIRB21, CIRB28, or a combination thereof.

### CLAIM OF PRIORITY

This application claims the benefit of U.S. Provisional Patent Application No. 63/181,025, filed on Apr. 28, 2021, which is incorporated by reference herein in its entirety.

### TECHNICAL FIELD

Described herein are immune cells (e.g., natural killer (NK), T cells) expressing a chimeric protein comprising IL2 and IL2Rβ (e.g., CIRB), a chimeric protein comprising IL2, IL2Rβ and IL21R (e.g., CIRB21), a chimera protein comprising IL2, IL2Rβ, and CD28 (e.g., CIRB28), or a combination thereof, and comprising a nucleic acid encoding a pore-forming protein, and methods of using such immune cells for treating a subject (e.g., a subject having cancer, graft-versus-host disease (GVHD), or an autoimmune disease. Expression of the pore-forming protein can be induced to promote destruction of immune cells expressing CIRB, CIRB21, CIRB28, or a combination thereof.

### BACKGROUND

Natural killer (NK) cells are lymphocytes endowed with the innate ability to attack malignant and virus infected cells without prior exposure to specific antigens (1-3). Several interleukins, and in particular IL2, activate and expand critical immune cells such as T-cells and NK cells (4). Systemic IL2 supplementation could therefore enhance immunity in a variety of diseases ranging from cancer to viral infection. However, in cancer patients, tumor cells and their microenvironment (TME) often repress NK cells anti-tumor activity by orchestrating a multitude of escape mechanisms (5).

Clinical trials using high dose IL2 infusions have met limited success due to severe side effects that mimic sepsis (6-8), while low-dose IL2 efficacy is limited by the short half-life (less than 10 min) of IL2 in vivo (9), and due to depletion of low IL2 doses by T-regs and other lymphoid cells (10). A number of strategies based on IL2 have aimed to enhance NK cytotoxicity while reducing toxicity in patients, with limited efficacy. Cultured ex-vivo NK cells can be activated and induced to proliferate by exposure to IL2 before transfer in vivo. Ex-vivo activated autologous NK cells display less anti-tumor efficacy (11) than NK cells from allogeneic donors (12), because self-class I HLA signaling suppresses NK cytotoxicity and cytokine release (13). However, in order for allogeneic donor NK cells to be effective, pre-transfer lymphodepletion to reduce competition for growth factors and cytokines is required (14,15). Moreover, systemic IL2 administration is needed to sustain NK cytotoxicity after in vivo transfer, exposing patients to systemic side effects.

Past therapeutic efforts to express endogenous IL2 in NK cells showed limited success with micro metastatic models and were not as efficacious as NK cells stimulated with exogenous IL2 (16). Similarly, effort to express membrane-bound endogenous IL2 did not show any advantage above parental NK92 cells (17). The limited success of several immunotherapy strategies using NK cells could be explained by the failure of activated NK cells to outcompete T-regs for cytokines in the host and the immunosuppressive effect of the TME, which includes myeloid derived suppressor cells (MDSCs). Both MDSCs and T-regs mediate NK cell functions suppression either by direct contact or by secretion of TGFβ1 (18, 19).

### SUMMARY

Interleukin-2 (IL2) is an immunostimulatory cytokine for key immune cells including T cells and natural killer (NK) cells. Systemic IL2 supplementation could enhance NK-mediated immunity in a variety of diseases ranging from neoplasms to viral infection. However, its systemic use is restricted by its serious side effects and its efficacy may be limited by activation of T-regulatory (T-regs) cells. IL2 signaling is mediated through interactions with a high affinity multi-subunit receptor complex containing IL2Rα, IL2Rβ and IL2Rγ. Adult NK cells may express only IL2Rβ and IL2Rγ subunits and are therefore relatively insensitive to IL2. To overcome these limitations, we created a novel chimeric IL2-IL2Rβ (CIRB) fusion protein of IL2 and its receptor IL2Rβ joined via a peptide linker. NK92 cells expressing CIRB (NK92^{CIRB}) are highly activated and expand indefinitely without exogenous IL2. They are highly cytotoxic, and were resistant to TGF-β1 and dexamethasone. Furthermore, CIRB induced substantial expression of natural cytotoxicity receptors NKP44, NKP46 and NKP30 as well as CD16, which enhanced NK cytotoxicity with Trastuzumab via antibody dependent pathways against HER2 positive cells. When compared to an IL2 secreting NK92 cell line (NK92^{IL2}), NK92^{CIRB} cells display superior in vivo anti-tumor effect and survival in mice (at least 3 weeks). This novel chimera eliminates the need for both IL2Rα and IL2Rβ expression and offers an alternative to exogenous IL2 stimulation. Collectively, it was shown that tethering IL2 to its receptor IL2Rβ offers a new platform that may be useful in selectively activating and enhancing immune therapy. See Publication No. U.S. 2020/0316118 A1, which is incorporated herein by reference in its entirety.

Provided herein is an improved platform that uses pore-forming proteins such as L-Holin to initiate destruction of immune cells tethering IL2 to its receptor IL2Rβ, thereby negating side effects that might occur in the patient due to prolonged exposure to the immune cells. Accordingly, immune cells described herein can be administered to a patient for a time sufficient to provide therapeutic effects to the patient, (e.g., a time sufficient to induce a reduction in tumor growth in a subject having cancer), and then the pore-forming protein can be expressed in the immune cell to induce its self-destruction in the patient.

Unless otherwise defined, all technical and scientific terms used berein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 includes diagrams of the Human IL2 and the chimera IL2 fused with receptor IL2Rβ (CIRB) in lentiviral constructs. A Linker (L) composed of the cMyc tag (EQKLISEEDL (SEQ ID NO: 29)) and a fragment of the extracellular domain of IL2 receptor alpha (EMETSQFPGEEKPQASPEGRPESETSC (SEQ ID NO: 28)), joins IL2 and its receptor IL2Rβ.

FIG. 2 includes an exemplary schematic of an IL2-IL2Rβ-CD28 chimera (CIRB28).

FIG. 3 includes an exemplary schematic of an IL2-IL2Rβ-IL21R chimera (CIRB21).

FIG. 4 includes an exemplary schematic of L-Holin and a chimeric cytokine receptor (e.g., CIRB, CIRB28) or a chimeric antigen receptor (CAR) in a lentiviral construct.

FIGS. 5A-5B includes data showing that L-Holin expression triggers death in proliferating glioblastoma GL261 (FIG. 5A) and NK92^{CIRB21} cells (FIG. 5B).

FIG. 6 includes a graph showing that L-Holin expression does not affect NK92^{CIRB21} cytotoxicity.

FIGS. 7A-7B include data comparing the effectiveness of L-Holin and icasp9 in triggering death of proliferating glioblastoma GL261.

FIGS. 8A-8B include graphs showing that GL261 cells (FIG. 7A) or NK92 cells (FIG. 7B) expressing icas9 grow more slowly than cells expressing L-Holin.

FIGS. 9A-9B include data showing that NK92 cells expressing icasp9 are viable at high doses of AP1093.

FIG. 10 includes a graph showing that EGFR-CAR improves the cytotoxicity of NK92^{CIRB21} cells.

FIG. 11 includes a graph showing that NK92^{CIRB21} cells are less sensitive than NK92^{CIRB} cells to lactate dehydrogenase inhibition by R-GNE140.

FIG. 12 includes a graph showing that NK92^{CIRB21} cells are more sensitive than NK92^{CIRB} cells to L-holin.

FIG. 13A includes a graph showing the fold increase of immune checkpoint proteins on NK92^{CIRB21} cells co-incubated with prostate cancer cells compared to untreated NK92^{CIRB21} cells.

FIG. 13B includes a graph showing absolute percent of NK92^{CIRB21} cells expressing immune checkpoint proteins.

### DETAILED DESCRIPTION

The present compositions and methods selectively activate and expand NK cells without exogenous IL2, while maintaining NK cytotoxicity and proliferation both in vitro and in vivo, circumvent the requirement of IL2Rα and its lack of expression in NK cells, thus avoiding IL2 off-target effects, cytokine competition, and activation of down-regulating lymphoid cells like T-regs.

IL2 will bind to either low affinity receptor IL2Rα (CD25) (21) or to intermediary affinity receptor IL2Rβ (CD122) with the common IL2Rγ chain (CD132) (22, 23) and to all, to form a high affinity quaternary complex(24). Adult NK cells may express only IL2Rβ and IL2Rγ subunits (25) and are, therefore, relatively insensitive to low doses of IL2, but acquire sensitivity upon IL2Rα expression (26). A recently developed IL2 “superkine” (27) that bypasses IL2Rα by binding directly and with high affinity to IL2Rβ produced better antitumor effects than wild type IL2 in mice. However, it still causes some form of pulmonary edema.

The novel chimera CIRB described herein comprises IL2 and its receptor IL2Rβ, joined by a peptide linker derived from the extracellular domain of IL2Rα. The linker was computationally determined as reasonably flexible, without adversely affecting the chimera stability which is generally inversely correlated to flexibility (28). When introduced in NK92 cells, CIRB induces indefinite cell expansion and conferred an in vitro cytotoxicity similar or higher than that elicited by IL2 expression. In vivo, the anticancer activity of NK92^{CIRB} against mid-size solid tumors was substantially superior to that elicited by NK92^{IL2}. Additionally, CIRB confers, in contrast to IL2, substantial resilience to TGFβ1, dexamethasone as well as IL4. This advantage could be crucial in the TME where TGFβ1 is secreted by a variety of cells including cancer associated fibroblasts (29), and exists in a membrane bound form on T-regs to induce anergy of NK cells (30), or by MDSCs to inhibit NKG2D expression, and IFN-γ production in NK cells (31). Cancer cells also regularly shed tumor-derived exosomes (TDEs) containing a membrane bound form of TGFβ1 resulting in the down regulation of NKG2D (32), and the inhibition of IL2 signaling (33). TGFβ1 mediates NK inhibition by an induced microRNA (miR)-183 which represses the co-activator/adapter DAP12 expression, thus destabilizing several activation signals in NK cells (34). CIRB expression in NK92^{CIRB} cells also provides resistance to dex while NK92^{IL2} cells were eliminated. Dex impairs the function of lymphocytes in part by suppressing IL2 production from CD4+T cells, and reducing the activation receptors NKG2D and Nkp46 in NK cells (35). Glucocorticoid hormones can interfere with macrophage activation and antigen presentation, repress the transcription of several pro-inflammatory cytokines, chemokines, cell adhesion molecules and other enzymes involved in the inflammatory response (36). The extreme sensitivity of NK92^{IL2} to dex, could be explained by the previously reported destabilization of IL2 RNA (37). This RNA destabilization could potentially occur in NK92^{IL2} cells but not when it is fused with IL2Rβ RNA as in NK92^{CIRB} cells.

CIRB and to a lesser degree the stable expression of IL2 allowed substantial CD16 expression in NK92 cell line. However, exogenous recombinant IL2 was not able to mediate such expression. Similarly, NK92-MI cell line which produces and secretes IL2 was found deficient in CD16, as previously reported (38). When combined with Trastuzumab, CD16 expression further enhanced NK92^{CIRB} and NK92^{IL2} cytotoxicity by ADCC. CIRB induced substantial expression of NCRs, NKP44 (9 fold), NKP46 (1.4 fold) and NKP30 (1.7 fold) as well as a modest but significant increase in INFγ. Finally, Granzyme-B expression declined substantially in NK92^{IL2}. Interestingly, CD25 expression declined dramatically in NK92^{CIRB}, as it is unnecessary in the presence of the chimera CIRB.

Current genetic modifications introducing CD16 in NK cells were shown to increase NK cell mediated ADCC against multiple myeloma when combined with Elotuzumab (39). The fact that CD16 was induced only in NK92^{CIRB} and NK92^{IL2} but not in NK92-MI or NK92 stimulated with IL2 could be possibly explained by the persistent IL2 signaling that somehow translates into stronger activation and growth NK92^{CIRB} and NK92^{IL2}. In fact, the growth rates of both NK92^{CIRB} and NK92^{IL2} were 2-fold that of NK92-MI, suggesting a higher level of activation. Another indication of higher activation of NK92^{CIRB} and NK92^{IL2} is the dramatic induction of NKP44, compared to parental NK92 stimulated with IL2 for 48 hours.

Additionally, NK92^{CIRB} can proliferate in vivo far longer and also have a better survival after irradiation than NK92^{IL2} cells. They also surpass that of NK92-MI when exposed to similar conditions (38) In vitro, NK92^{IL2} cells secrete sufficient IL2 to sustain their activation and proliferation. However, they may not be able to produce enough IL2 extracellular concentrations to sustain activation and proliferation in vivo. This could be compounded by the competition for IL2 by T-regs and other immune cells in an immune competent animal.

Thus the novel chimeras described herein comprising CIRB endow NK92 cells with very useful attributes that improve immune therapy of cancer and potentially viral infections.

Cellular immunotherapy using donor NK cells is an emerging field that could achieve significant anti-cancer effects, safely and without the risk of inducing graft-versus-host disease (GVHD). This safety feature as well as the off tumor/on target toxicity are currently hindering the success of CAR-T technology (40). Several NK cell lines (Khyg-1, NKL, NKG, NK-YS, YT, YTS and HANK-1 cells) are currently used in preclinical studies. However, only the NK92 cell line has been extensively evaluated for its safety and efficacy in clinical settings (41, 42). NK92 cells are CD56³⁰ , CD3⁻ and CD16⁻ and require IL2 for growth and activation (43). Unlike primary NK cells, NK92 cells and other NK cell lines constitute a stable and homogenous population. They are amenable to genetic modification by lentiviruses, a gene transfer platform that has shown a good safety profile for lymphocytes (44). Many encouraging advances have been achieved in NK cell-directed immunotherapy (45). However, the increasing demand for NK cells expansion ex-vivo requires both highly activated cells and reduced costs of cell expansion. Moreover, infused cells must have higher activation potential and possess favorable characteristics against immunosuppressors found in the TME.

The present strategy includes fusing interleukins to their receptors in the CIRB, CIRB28, and CIRB21 chimeras achieves better cytokine activation, with specificity, and without systemic toxicity or competition by other cellular components of the immune system. Self-activation of NK cells provides several distinguishing features such as resilience to TGFβ1 or glucocorticoid hormones, substantial expression of CD16, higher survival after irradiation and a superior antitumor activity in vivo.

### Chimeric Proteins

The present disclosure provides chimeras as described herein, e.g., a chimera comprising IL2 and IL2Rβ (e.g., CIRB); a chimera comprising IL2, IL2Rβ and IL21R (e.g., CIRB21); and/or a chimera comprising IL2, IL2Rβ, and CD28 (e.g., CIRB28). All of the fusion proteins described herein can be generated using standard molecular biological procedures, e.g., for manipulating and expressing recombinant DNA. See, e.g., Current Protocols in Molecular Biology, Ausubel, F. M. et al. (eds.) John Wiley & Sons (1995), and Green and Sambrook, Molecular Cloning: A Laboratory Manual (Fourth Edition), Cold Spring Harbor Laboratory Press (Jun. 15, 2012) and supplements thereof, and other standard laboratory manuals. The chimeras can be expressed, e.g., stably expressed, in an NK cell, e.g., a primary or cultured NK cell. The cells are then infused into a subject, e.g., a subject who has (e.g., has been diagnosed with) cancer.

Non-limiting examples of chimeras that can be used as described herein are provided in Publication No. U.S. 2020/0316118 A1, which is incorporated herein by reference in its entirety.

Provided herein are exemplary sequences for the various domains that make up the chimeras described herein. In some embodiments, the sequences used are at least 80% identical to the exemplary sequence as defined herein. In some embodiments, the sequences are at least 85%, 90%, 95%, 99% or 100% identical.

To determine the percent identity of two sequences, the sequences are aligned for optimal comparison purposes (gaps are introduced in one or both of a first and a second amino acid or nucleic acid sequence as required for optimal alignment, and non-homologous sequences can be disregarded for comparison purposes). The length of a reference sequence aligned for comparison purposes is at least 80% (in some embodiments, about 85%, 90%, 95%, or 100% of the length of the reference sequence) is aligned. The nucleotides or residues at corresponding positions are then compared. When a position in the first sequence is occupied by the same nucleotide or residue as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package, using a Blossum 62 scoring matrix with a gap penalty of 12, a gap extend penalty of 4, and a frameshift gap penalty of 5.

### IL2-IL2Rβ (CIRB)

The fusion proteins described herein include, inter alia, IL2 and IL2Rβ fused together with an intervening linker. Sequences for IL2 are known in the art; an exemplary human IL2 precursor sequence is shown in SEQ ID NO: 34.

| (SEQ ID NO: 34) | |
|---|---|
| 1 | MYRMQLLSCI ALSLALVTNS APTSSSTKKT QLQLEHLLLD LQMILNGINN YKNPKLTRML |
| 61 | TFKFYMPKKA TELKHLQCLE EELKPLEEVL NLAQSKNFHL RPRDLISNIN VIVLELKGSE |
| 121 | TTFMCEYADE TATIVEFLNR WITFCQSIIS TLT |

Amino acids 1-20 are a signal sequence, and so can be replaced by other signal sequences if desired.

Linker sequences known in the art can be used between the various domains of the fusion protein; for example, one, two, three, four, five or more GGGS sequences can be used. In preferred embodiments, the linker between IL2 and the N-terminus of IL2Rβ comprises the extracellular domain of IL2Rα (EMETSQFPGEEKPQASPEGRPESETSC (SEQ ID NO: 28). A tag, e.g., a cMyc tag (EQKLISEEDL (SEQ ID NO: 29)), can also be added, e.g., between IL2 and the linker. An exemplary nucleic acid sequence encoding IL2 is available in GenBank at Acc. No. NM_000586.3.

Sequences for IL2Rβ are also known in the art; an exemplary human IL2Rβ precursor sequence is shown in SEQ ID NO: 35.

| (SEQ ID NO: 35) | |
|---|---|
| 1 | MAAPALSWRL PLLILLLPLA TSWASAAVNG TSQFTCFYNS RANISCVWSQ DGALQDTSCQ |
| 61 | VHAWPDRRRW NQTCELLPVS QASWACNLIL GAPDSQKLTT VDIVTLRVLC REGVRWRVMA |
| 121 | IQDFKPFENL RLMAPISLQV VHVETHRCNI SWEISQASHY FERHLEFEAR TLSPGHTWEE |
| 181 | APLLTLKQKQ EWICLETLTP DTQYEFQVRV KPLQGEFTTW SPWSQPLAFR TKPAALGKDT |
| 241 | IPWLGHLLVG LSGAFGFIIL VYLLINCRNT GPWLKKVLKC NTPDPSKFFS QLSSEHGGDV |
| 301 | QKWLSSPFPS SSFSPGGLAP EISPLEVLER DKVTQLLLQQ DKVPEPASLS SNHSLTSCFT |
| 361 | NQGYFFFHLP DALEIEACQV YFTYDPYSEE DPDEGVAGAP TGSSPQPLQP LSGEDDAYCT |
| 421 | FPSRDDLLLF SPSLLGGPSP PSTAPGGSGA GEERMPPSLQ ERVPRDWDPQ PLGPPTPGVP |
| 481 | DLVDFQPPPE LVLREAGEEV PDAGPREGVS FPWSRPPGQG EFRALNARLP LNTDAYLSLQ |
| 541 | ELQGQDPTHL V |

Amino acids 1-26 are a signal sequence, and are preferably deleted in the present constructs, e.g., the sequence comprises amino acids 27-551 of SEQ ID NO: 35. Exemplary nucleic acid sequences encoding IL2Rβ are available in GenBank at Acc. No. NM_000878.4 (Var. 1), NM_001346222.1 (Var. 2); and NM_001346223.1 (Var. 3). Variants 1, 2 and 3 encode the same protein.

### IL2-IL2Rβ-IL-21 (CIRB21)

Interleukins IL4, IL7, IL9, IL15 and IL21 belong to the same family as IL2, and use the same common IL2Rg. They all have their own private receptors, except for IL2 and IL15, which use IL2Rβ in addition to their own alpha receptors. When soluble IL2, IL4, IL7, or IL21 were added to NK92 cells expressing the chimera NK92^{CIRB}, only IL21 dramatically enhance cytotoxicity against PC-3 cells. Thus the entire cytoplasmic domain of IL21R was cloned then added Head-to-Tail to the C-terminal of IL2Rβ in the chimera CIRB. This resulted in a novel IL2-IL2Rβ-IL21R chimera (called CIRB21). As shown herein, it was possible to emulate the activation signals from multiple cytokines that activate NK cells via different receptors by using only one ligand and a hybrid receptor.

In some embodiments, the present constructs include the cytoplasmic domain of IL21R at the C-terminus of the IL2Rβ portion (optionally with an intervening linker therebetween). Sequences for IL21R are also known in the art; an exemplary human IL21R precursor sequence is shown in SEQ ID NO: 36.

| (SEQ ID NO: 36) | |
|---|---|
| 1 | MPRGWAAPLL LLLLQGGWGC PDLVCYTDYL QTVICILEMW NLHPSTLTLT WQDQYEELKD |
| 61 | EATSCSLHRS AHNATHATYT CHMDVFHFMA DDIFSVNITD QSGNYSQECG SFLLAESIKP |
| 121 | APPFNVTVTF SGQYNISWRS DYEDPAFYML KGKLQYELQY RNRGDPWAVS PRRKLISVDS |
| 181 | RSVSLLPLEF RKDSSYELQV RAGPMPGSSY QGTWSEWSDP VIFQTQSEEL KEGWNPHLLL |
| 241 | LLLLVIVFIP AFWSLKTHPL WRLWKKIWAV PSPERFFMPL YKGCSGDFKK WVGAPFTGSS |
| 301 | LELGPWSPEV PSTLEVYSCH PPRSPAKRLQ LTELQEPAEL VESDGVPKPS FWPTAQNSGG |
| 361 | SAYSEERDRP YGLVSIDTVT VLDAEGPCTW PCSCEDDGYP ALDLDAGLEP SPGLEDPLLD |
| 421 | AGTTVLSCGC VSAGSPGLGG PLGSLLDRLK PPLADGEDWA GGLPWGGRSP GGVSESEAGS |
| 481 | PLAGLDMDTF DSGFVGSDCS SPVECDFTSP GDEGPPRSYL RQWVVIPPPL SSPGPQAS |

Preferably, in these embodiments the IL21R-derived domain comprises amino acids 254-538 of SEQ ID NO: 36. An exemplary nucleic acid sequences encoding IL21R is available in GenBank at Acc. No. NM_021798.3.

### IL2-IL2Rβ-CD28 (CIRB28) in NK Cells

NK cells (and others) are activated when MHC-1 molecule expression is down regulated in transformed cells (Algarra et al., Hum Immunol 2000; 61(1):65-73) and during viral infection (Tortorella et al., Annu Rev Immunol 2000; 18:861-92). However, the acquisition of resistance phenotype by tumor cells is often caused by the expression of inhibitory signals from MHC-1 (Kochan et al., Oncoimmunology 2013; 2(11):e26491). HLA-G in particular is known to inhibit NK92 mediated tumor cell lysis (Lin et al., Ann Oncol 2007; 18(11):1804-9). One potential solution to this problem could be the use of multiple activating signals to offset these inhibitory signals. Among the most effective co-stimulatory molecules used for T-cells are CD28 and 4-1BB. CD28 activation requires CD80 and CD86 stimulatory ligand expression on tumor cells. As a result, CD80 expression in tumors was shown to lead to their rejection (Townsend et al., Science 1993; 259(5093): 368-70), conversely, in CD28^{-/-} mice, cellular and T cell-dependent immunity are quite deficient (Shahinian et al., Science 1993; 261(5121):609-12). Therefore, low levels of CD80 are considered an escape mechanism for tumors in several cancers (Tirapu et al., Cancer Res 2006; 66(4): 2442-50; Hersey et al., Int J Cancer 1994; 58(4): 527-32; Bernsen et al., Br J Cancer 2003; 88(3):424-31). For example, the use of a CD28 activation domain in an anti erbB2 chimeric receptor allowed the inhibition of tumor progression in vivo of a MHC-1⁺ lymphoma Pegram et al., J Immunol 2008; 181(5):3449-55). Although CD28 is expressed by NK92 cells (Gong et al., Leukemia 1994; 8(4):652-8), its activation is not mediated by all cancers.

In some embodiments, the present constructs include the activation domain of CD28 at the C-terminus of the IL2Rβ portion (optionally with an intervening linker therebetween). Sequences for CD28 are also known in the art; an exemplary human CD28 precursor sequence is shown in SEQ ID NO: 38.

| (SEQ ID NO: 38) | |
|---|---|
| 1 | MLRLLLALNL FPSIQVTGNK ILVKQSPMLV AYDNAVNLSC KYSYNLFSRE FRASLHKGLD |
| 61 | SAVEVCVVYG NYSQQLQVYS KTGFNCDGKL GNESVTFYLQ NLYVNQTDIY FCKIEVMYPP |
| 121 | PYLDNEKSNG TIIHVKGKHL CPSPLFPGPS KPFWVLVVVG GVLACYSLLV TVAFIIFWVR |
| 181 | SKRSRLLHSD YMNMTPRRPG PTRKHYQPYA PPRDFAAYRS |

Preferably, in these embodiments the CD28-derived domain comprises the intracellular domain, e.g., amino acids 180 to 220 of SEQ ID NO: 38. i.e., RSKRSRLLHSDYMNMTPRRPGPTRKHYQPYAPPRDFAAYRS (SEQ ID NO: 39). An exemplary nucleic acid sequence encoding CD28 is available in GenBank at Acc. No. NM_006139.3.

### IL2-IL2Rβ-CD28 (CIRB28) in Regulatory T Cells (T-Regs)

Patients with hematological malignancies greatly benefit from allogeneic bematopoietic stem cell transplant (AHSCT). In this strategy the donor immune system will attack the patient tumor cells with curative potential in a phenomena known as graft versus tumor. Unfortunately, the donor immune cells may also attack the recipient patient healthy tissue either immediately or in the 100 days that follows and cause GVHD. This could lead to death in 15% and/or morbidity in 40 to 60% of AHSCT. Immuno-suppression is currently the standard of care to manage GVHD (Luznik and Fuchs, Immunol Res 2010; 47(1-3):65-77; Storb et al., Biol Blood Marrow Transplant 2010; 16(1 Suppl):S18-27). However, T-regs cells expressing the transcription factor Forkhead box P3 (FOXP3) (Roncador et al., Eur J Immunol 2005; 35(6): 1681-91; Hall et al., J Exp Med 1990; 171(1): 141-57) have been found to suppress or alleviate GVHD during AHSCT (Beres et al., J Immunol 2012; 189(1): 464-74; Brunstein et al., Blood 2011; 117(3): 1061-70). The persistence of FOXP3 expression is maintained by the epigenetic demethylation of 11 CpG motifs in the conserved non-coding sequence 2 (CNS2), located in its first intron. This demethylation pattern lasts for the life span of T-regs and is protected by Ten-Eleven-Translocation DNA dioxygenase, which is recruited to CNS2 by STATS activated by IL2 signaling (Nair et al., Mol Cells 2016; 39(12):888-97), to protect the CpG motifs in CNS2 from re-methylation by DNA methyltransferases. Similarly, CTLA-4, an important down regulator of T-cell activation is up regulated in T-regs and is also controlled by IL2 (Wang et al., Scand J Immunol 2001; 54(5): 453-8; Bell et al., J Autoimmun 2015; 56:66-80; Gasteiger et al., Front Immunol 2012; 3:179). T-regs are extremely responsive to IL2, due to their massive CD25 expression (Dieckmann et al., Exp Med 2001; 193(11): 1303-10) and their ability to reach IL2 sources by chemokine receptor CCR7 (Smigiel et al., J Exp Med 2014; 211(1):121-36). However, activated T-regs have been shown to lower CD25 expression and change their IL2 signaling in favor of ICOS signaling pathway. This leads to instability of FOXP3 expression making the transition possible from an activated and not terminally differentiated T-regs (Sharma et al., Immunity 2010; 33(6): 942-54) to a pro-inflammatory T-cell effector or develop into IFN-gamma-producing pro-inflammatory Th1 effector cells (Zhang et al., J Immunol 2017; 198(7):2612-25; Feng et al., Gastroenterology 2011; 140(7):2031-43; Takahashi et al., J Exp Med 2011; 208(10):2055-67) or even Th17 (46). In short, T-regs long-term activation and demethylation of CNS2 as well as proliferation require both IL2 and CD28 co-stimulations (Tang and Bluestone, Immunol Rev 2006; 212:217-37; Chen et al., J Immunol 2011; 186(11):6329-37).

Thus, the IL2-IL2Rβ-CD28 chimeras described herein could have a dual use: to help NK92 cells override inhibitory signals from MHC-1+cancer cells, and separately, to activate T-regs for the purpose of treating GVHD. As described herein, without wishing to be bound by theory, addition of the activation domain of CD28 into a novel chimera, IL2-IL2Rβ-CD28, combining co-stimulatory signals from IL2 and CD28 will lead to a superior NK92 activation that could help override tumor escape via MHC-1+. This chimera could also lead to proliferation of T-regs cells with long-term FOXP3 expression. This strategy could bypass the use of artificial antigen presenting cells (aAPC), dendritic cells or anti-CD3 antibody required for T-regs activation and expansion.

### Pore-Forming Proteins

The present disclosure provides pore-forming proteins that form pores or channels in the membrane of a cell, thereby killing the cell. As used herein, the term “pore-forming protein” refers to any protein capable of disrupting the cell membrane and inducing cell death when produced inside a cell.

The pore-forming proteins for use in methods and compositions described herein can be derived from bacteria or viruses. For example, the pore-forming protein can be a holin protein such as L-Holin from bacteriophage. Non-limiting examples of holin proteins for use in methods and compositions described herein include L-Holin, T4t holin, P21 holin, P2 holin, P35 holin, T7 holin, HP1 holin, and T4 holin. See also Kuppusamykrishnan et al., Analysis of 58 Families of Holins Using a Novel Program, PhyST. J Mol Microbiol Biotechnology 2016; 26:381-388, the relevant disclosures of which are incorporated by reference herein for the subject matter and purpose referenced herein.

In some embodiments, the pore-forming protein comprises L-Holin. An example of an amino acid sequence of L-Holin is provided below:

(SEQ ID NO: 1)
MPEKHDLLAAILAAKEQGIGAILAFAMAYLRGRYNGGAFTKTVIDATMC
AIIAWFIRDLLDFAGLSSNLAYITSVFIGYIGTDSIGSLIKRFAAKKAG
VEDGRNQ

The pore-forming protein can comprise full-length L-Holin or a fragment thereof. In some embodiments, the pore-forming protein comprises an amino acid sequence having at least 80%, at least 85%, at least 90%, at least 95%, or at least 99% identity to L-Holin.

In some embodiments, the pore forming protein is L-Holin.

### Nucleic Acids and Expression Vectors

The compositions described herein can include nucleic acid molecules encoding a chimera, a pore-forming protein, or both as described herein. Nucleic acid molecules comprising expression vectors can be used for expression of the chimeras, e.g., in an immune cell such as an NK or T-reg cell as described herein.

A nucleic acid encoding the selected chimera and/or pore-forming protein can be inserted in an expression vector, to make an expression construct. The nucleic acid encoding the chimera and the nucleic acid encoding the pore-forming protein can be inserted into the same expression vector or different expression vectors. A number of suitable vectors are known in the art, e.g., viral vectors including recombinant retroviruses, adenovirus, adeno-associated virus, lentivirus, herpes simplex virus-1, adenovirus-derived vectors, or recombinant bacterial or eukaryotic plasmids. For example, the expression construct can include a coding region for the chimera and one or more regulatory regions, e.g., a promoter sequence, e.g., a promoter sequence that restricts expression to a selected cell type, a conditional promoter, or a strong general promoter; an enhancer sequence; untranslated regulatory sequences, e.g., a 5′untranslated region (UTR), a 3′UTR; a polyadenylation site; and/or an insulator sequence, that direct expression of the chimera. Such sequences are known in the art, and the skilled artisan would be able to select suitable sequences. See, e.g., Current Protocols in Molecular Biology, Ausubel, F. M. et al. (eds.) John Wiley & Sons (1995), and Green and Sambrook, Molecular Cloning: A Laboratory Manual (Fourth Edition), Cold Spring Harbor Laboratory Press (Jun. 15, 2012) and supplements thereof, and other standard laboratory manuals.

In some examples, the one or more regulatory regions comprises an inducible promoter. As used herein, the term “inducible promoter” refers to a control element (e.g., a promoter, enhancer, promoter/enhancer, or portion thereof) whose transcriptional activity may be regulated by exposing a cell comprising a nucleic acid sequence operably linked to the promoter to a treatment or condition that alters the transcriptional activity of the promoter, resulting in increased transcription of the nucleic acid sequence. In some examples, the term “inducible promoter” also includes repressible promoters, i.e., promoters whose transcriptional activity may be regulated by exposing a cell comprising a nucleic acid sequence operably linked to the promoter to a treatment or condition that alters the transcriptional activity of the promoter, resulting in decreased transcription of the nucleic acid sequence. Non-limiting examples of an inducible promoter or a promoter system include, but are not limited to, a tetracycline-dependent regulatory system, an ecdysone inducible promoter (EcP), a T7 promoter/T7 RNA polymerase system (T7P), a glucocorticoid responsive mouse mammary tumour virus (MMTV) promoter, a steroid-inducible promoter such as a promoter for a gene encoding a glucocorticoid or estrogen receptor (inducible by treatment with the corresponding hormone), a metallothionine promoter (inducible by treatment with various heavy metals), a MX-1 promoter (inducible by interferon), a “GeneSwitch” mifepristone-regulatable system (Sirin et al., 2003, Gene, 323:67), and a cumate inducible gene switch (WO 2002/088346).

In some examples, one or more regulatory regions comprises a tissue-specific promoter to achieve tissue-specific expression of a nucleic acid sequence, e.g., to achieve expression of a nucleic acid sequence in tissue affected by cancer. Examples of tissue specific promoters include, but are not limited to: an B29 promoter (B cell expression), a runt transcription factor (CBFa2) promoter (stem cell specific expression), an CD 14 promoter (monocytic cell expression), an CD43 promoter (leukocyte and platelet expression), an CD45 promoter (hematopoietic cell expression), an CD68 promoter (macrophage expression), a CYP450 3A4 promoter (hepatocyte expression), an desmin promoter (muscle expression), an elastase 1 promoter (pancreatic acinar cell expression, an endoglin promoter (endothelial cell expression), a fibroblast specific protein 1 promoter (FSP1) promoter (fibroblast cell expression), a fibronectin promoter (fibroblast cell expression), a fms-related tyrosine kinase 1 (FLT1) promoter (endothelial cell expression), a glial fibrillary acidic protein (GFAP) promoter (astrocyte expression), an insulin promoter (pancreatic beta cell expression), an integrin, alpha 2b (ITGA2B) promoter (megakaryocytes), an intracellular adhesion molecule 2 (ICAM-2) promoter (endothelial cells), an interferon beta (IFN-β) promoter (hematopoietic cells), a keratin 5 promoter (keratinocyte expression), a myoglobin (MB) promoter (muscle expression), a myogenic differentiation 1 (MYOD1) promoter (muscle expression), a nephrin promoter (podocyte expression), a bone gamma-carboxyglutamate protein 2 (OG-2) promoter (osteoblast expression), an 3-oxoacid CoA transferase 2B (Oxet2B) promoter, (haploid-spermatid expression), a surfactant protein B (SP-B) promoter (lung expression), a synapsin promoter (neuron expression), and a Wiskott-Aldrich syndrome protein (WASP) promoter (hematopoietic cell expression).

Expression constructs can be administered in any biologically effective carrier, e.g., any formulation or composition capable of effectively delivering the component gene to cells in vivo. Viral vectors transfect cells directly; plasmid DNA can be delivered with the help of, for example, cationic liposomes (e.g., Lipofectin) or derivatized (e.g., antibody conjugated), polylysine conjugates, gramicidin S, artificial viral envelopes or other such intracellular carriers, as well as direct injection of the gene construet or CaPO₄ precipitation. In some embodiments, the nucleic acid is applied “naked” to a cell, i.e., is applied in a simple buffer without the use of any additional agents to enhance uptake. See, e.g., Current Protocols in Molecular Biology, Ausubel, F. M. et al. (eds.) Greene Publishing Associates, (1989), Sections 9.10-9.14 and other standard laboratory manuals.

### Immune Cells

The present methods include expressing, either stably or transiently, a chimera and a pore-forming protein described herein in an immune cell such as a NK cell (e.g., a CD3-CD56+lymphocyte; see Cheng et al., Cellular & Molecular Immunology (2013) 10, 230-252) or a T-reg cell. The NK cell can be a primary cell, e.g., derived from the peripheral blood of a subject and proliferated ex vivo, or can be a cultured NK cell.

In some examples, methods include expressing, either stably or transiently, a chimera described herein in an immune cell for a time sufficient to provide a therapeutic effect to a subject (e.g., a time sufficient to induce a reduction in tumor growth in a subject having cancer), and then expressing a pore-forming protein described herein in the immune cell to induce self-destruction of the immune cell. As such, the number of immune cells is reduced or eliminated in a patient, thereby preventing unwanted toxic effects that might occur from prolonged exposure to the immune cells.

When primary cells are used, allogeneic NK cells are preferred, as they were not exposed to immunosuppression and should be fully active. In preferred embodiments, the cells are obtained by performing apheresis on haploidentical related donors to collect peripheral blood leukocytes, which are then depleted of CD3+cells before optional expansion and administration. See, e.g., Davis et al., Cancer J. 2015 November-December; 21(6): 486-491. Alternatively, the cells can be obtained from peripheral or cord blood cells, stem cells or even induced pluripotent stem cells (iPSCs); see Cheng et al., Cellular & Molecular Immunology (2013) 10, 230-252.

Cultured NK cell lines are known in the art, e.g., including NK-92, KHYG-1, NKL, NKG, NK-YS, YT, YTS and haNK-1 cells, as are methods of making new NK cell lines. NK-92 is a cytolytic cancer cell line that was immortalized ex vivo from NK cells from the blood of a subject suffering from a non-Hodgkins lymphoma. NK-92 cells retain most of the activating receptors and cytolytic signaling pathways but lack the major inhibitory receptors displayed by normal NK cells, and do not express the Fe receptor CD16, and so cannot mediate antibody-dependent cellular cytotoxicity (ADCC). NK-92 cells are tumor-selective and non-immunogenic in humans. The NK-92 cell line is described in Gong et al., Leukemia. 8:652-8 (1994); Yan et al., Clin Cancer Res. 4:2859-68 (1998): WO1998/49268 and U.S. 2002/0068044. NK-92 cells have been evaluated for potential therapeutic use in cancers, including hematological malignancies; see, e.g., Ljunggren and Malmberg, Nat Rev Immunol. 2007 May; 7(5):329-39; Tonn et al., J Hematother Stem Cell Res. 2001 August; 10(4):535-44; Klingemann, Cytotherapy. 2005; 7(1):16-22; Malmberg et al., Cancer Immunol Immunother. 2008 October; 57(10):1541-52 haNK is an NK-92 variant cell line that expresses the high-affinity Fe receptor FcγRIIIa (158V), and is in clinical development to be combined with IgG1 monoclonal antibodies (mAbs). taNKs are targeted NK-92 cells that have been transfected with a gene that expresses a chimeric antigen receptor for a given tumor antigen. KHYG-1 cells were developed the blood of a patient with aggressive NK leukemia (Yagita et al., Leukemia (2000) 14, 922-930) that is IL-2 dependent and produces granzyme M. NKL cells were established from the peripheral blood of a patient with CD3-CD16+CD56+large granular lymphocyte (LGL) leukemia (Robertson et al., Exp Hematol. 1996 February; 24(3):406-15). NKG cells were established from the peripheral blood of a patient with rapidly progressive non-Hodgkin's lymphoma (Cheng et al., Cell Transplant. 2011; 20(11-12):1731-46). NK-YS cells were established from a patient with a leukemic-state nasal angiocentric natural killer (NK) cell lymphoma with systemic skin infiltration (Tsuchiyama et al., Blood. 1998 Aug. 15; 92(4):1374-83). YT cells, a human NK-like leukaemia cell line. was established from cells in the pericardial fluid of a patient with acute lymphoblastic lymphoma (ALL) and thymoma (Yodoi et al., J Immunol 134: 1623-1630 (1985)); Hamack et al., Anticancer Research 31(2):475-479 (2011)). YTS is a sub-clone of the NK cell leukemia line YT. All of these cell lines are commercially available. For additional information on NK cell lines, Klingermann et al., Front. Immunol. 7:91 (2016); Dahlberg et al., Front. Immunol. 6:605 (2015). The cells can be used as is, or modified, e.g., genetically modified as described in U.S. Pat. Nos. 7,618,817; 8,034,332 (NK-92 cells secreting cytokines including IL2); U.S. Pat. No. 8,313,943 (NK-92 cells expressing CD16); WO 2015193411 (CAR-expressing nk-92 cells); and WO2016160602 (NK-92 cells expressing FcR including CD16). Additional methods for generating and manufacturing cultured NK cells are known in the art; see, e.g., Chabannon et al., Front Immunol. 2016; 7: 504, which provides exemplary parameters for media, cytokines, and culture systems, inter alia.

The present methods also include expressing, either stably or transiently, a chimera described herein (e.g., IL2-IL2Rγ-CD28 chimera) in a T-reg cell, i.e., a CD4+/CD25+T cell. The T-reg cell can be a primary cell, e.g., derived from the peripheral blood of a subject and proliferated ex vivo, or can be a cultured T-reg cell.

When primary T-reg cells are used, ex vivo expanded donor T-reg cells, e.g., naturally occurring regulatory T cells (nT-regs) from peripheral blood, are preferred. In preferred embodiments, the cells are obtained from peripheral blood from a donor and expanded ex-vivo using methods known in the art; see, e.g., Dieckmann et al., J. Exp. Med. 193(11):1303-1310 (2001) Chakraborty et al., Haematologica 98(4):533-537 (2013); Hippen et al., Sci Transl Med. 2011 May 18; 3(83): 83ra41; and Taylor et al., Blood. 2002; 99:3493-3499. Alternatively, the cells can be obtained from umbilical cord blood (see, e.g., Brunstein et al., Blood. 2011 Jan. 20; 117(3): 1061-1070).

The NK and T-reg cells should be maintained according to good manufacturing practice (GMP) in GMP facilities

The NK or T-reg cells expressing a chimera as described herein, as well as any supplemental active agents for coadministration, can be incorporated into pharmaceutical compositions. Such compositions typically comprise the cells and a pharmaceutically acceptable carrier. A “pharmaceutically acceptable carrier” includes any and all solvents, antibacterial and antifungal agents, isotonic agents, and the like, compatible with pharmaceutical administration (Gennaro, 2000). Preferred examples of such carriers or diluents include, but are not limited to, water, saline, Ringer's solutions, dextrose solution, and 5% human serum albumin. Liposomes and non-aqueous vehicles such as fixed oils may also be used. Supplementary active compounds can also be incorporated into the compositions.

Sterile injectable solutions can be prepared by incorporating the active compound (e.g., NK or T-reg cells as described herein) in the required amount in an appropriate solvent with one or a combination of ingredients as required; preferably the solvent is already sterilized, or the formulation can be followed by sterilization. In some embodiments, the cells are cryopreserved.

### Cancer Immunotherapy

The methods described herein include methods for the treatment of disorders associated with abnormal apoptotic or differentiative processes, e.g., cellular proliferative disorders or cellular differentiative disorders, e.g., cancer, including both solid tumors and hematopoietic cancers. In some embodiments, the disorder is a solid tumor, e.g., breast, prostate, pancreatic, brain, hepatic, lung, kidney, skin, or colon cancer. Generally, the methods include administering a therapeutically effective amount of NK cells expressing a chimera as described herein, to a subject who is in need of, or who has been determined to be in need of, such treatment.

As used in this context, to “treat” means to ameliorate at least one symptom of the disorder associated with abnormal apoptotic or differentiative processes. For example, a treatment can result in a reduction in tumor size or growth rate. Administration of a therapeutically effective amount of a compound described herein for the treatment of a condition associated with abnormal apoptotic or differentiative processes will result in a reduction in tumor size or decreased growth rate, a reduction in risk or frequency of reoccurrence, a delay in reoccurrence, a reduction in metastasis, increased survival, and/or decreased morbidity and mortality, inter alia.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a discase state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers. renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoictic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

### GVHD and Autoimmune Disease

The methods described herein include methods the treatment of disorders associated with abnormal immune response, e.g., graft-versus-host disease or GVHD or autoimmune discase. The methods can include administering T-regulatory cells expressing a IL2-IL2Rβ-CD28 chimera as described herein to a subject who is in need thereof.

Allogeneic bone marrow transplantation (BMT) has been shown to be effective in hematologic malignancies and some solid tumors, but the high incidence of GVHD has limited the effectiveness and use of BMT. T-regs cells have shown efficacy in suppressing GVHD; see Olson et al., Blood. 2010 May 27; 115(21):4293-301; Sung and Chao, STEM CELLS TRANSLATIONAL MEDICINE, 2013; 2:25-32; Dieckmann et al., J. Exp. Med. 193(11): 1303-1310 (2001) Chakraborty et al., Haematologica 98(4):533-537 (2013); Hippen et al., Sci Transl Med. 2011 May 18; 3(83): 83ra41; Taylor et al., Blood. 2002; 99:3493-3499; and Brunstein et al., Blood. 2011; 117(3): 1061-1070).

Impairment of T-regs functions or resistance of effector T cells to T-regs has been reported in many autoimmune diseases such as type-1 diabetes (T1D) (Brusko et al., Diabetes 2005; 54(5):1407-14), rheumatoid arthritis (van Amelsfort et al., Arthritis Rheum 2004; 50(9):2775-85), multiple sclerosis (Fletcher et al., J Immunol 2009; 183(11):7602-10). systemic lupus erythematosus (Lyssuk et al., Adv Exp Med Biol 2007; 601:113-9) and psoriasis (Sugiyama et al., J Immunol 2005; 174(1):164-73), as well as atopic disease (Singer et al., Front Immunol. 2014; 5: 46). Elevated CD25 expression in T-regs makes them particularly responsive to IL2 and this was exploited for example in the case of T1D, where administration of low dose IL-2 promoted T-regs survival and protects NOD mice against diabetes (Tang et al., Immunity 2008; 28(5):687-9; Grinberg-Bleyer et al., J Exp Med 2010; 207(9):1871-8), and an infusion of T-regs preserved beta-cell function in type 1 diabetes in children (Marek-Trzonkowska et al., Diabetes Care (2012) 35:1817-2010).

T-regs, e.g., CD4+CD25+, e.g., CD4+CD25+CD127-Tregs (e.g., CD4+CD25^{high}CD127-ICOS+ for atopy Tregs or CD4+CD25+CD127-CD62L⁺ for GVHD), which are optionally FOXP3+ as well, expressing the IL2-IL2Rβ-CD28 chimeras can be used to reduce alloreactive T cells that are believed to mediate GVHD and autoimmunity and damage host tissues. In these embodiments, an effective amount of T-regs cells expressing a IL2-IL2Rβ-CD28 chimera as described herein is an amount sufficient to decrease numbers of alloreactive T cells and decrease the self-immune response, e.g., by reduction of donor T cell proliferation and increased T cell apoptosis. See, e.g., Singer et al., Front Immunol. 2014; 5: 46; Riley et al., Immunity. 2009 May; 30(5): 656-665.

### Methods of Administration and Dosing

The methods include administration, preferably by intravenous infusion, of a therapeutically effective amount of the immune cells (e.g., NK cells) described herein. A therapeutically effective dose can be determined empirically, e.g., based on animal experiments and clinical studies. In some embodiments, the methods include one or more infusions of at least 10⁴ and up to 1×10⁶, 5×10⁶, 1×10⁷, 5×10⁷, 1×10⁸, 3×10⁸, 5×10⁸, 1×10⁹, or 5×10⁹ cells per dose, e.g., between 1 billion and 3 billion cells. or any ranges between any two of the numbers, end points inclusive. The cells can be administered to a subject once or can be administered multiple times, e.g., once every 1, 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10 or more weeks during therapy, or any ranges between any two of the numbers, end points inclusive. See, e.g., Ljunggren and Malmberg, Nat Rev Immunol. 2007 May; 7(5):329-39; Tonn et al., J Hematother Stem Cell Res. 2001 August; 10(4):535-44; Klingemann, Cytotherapy. 2005; 7(1):16-22; Malmberg et al., Cancer Immunol Immunother. 2008 October; 57(10):1541-52; Cheng et al., Cellular & Molecular Immunology (2013) 10, 230-252.

In preferred embodiments, before being infused into a subject the cells are treated so that they are no longer capable of proliferating, but retain cytotoxic activity. One way of achieving this state is by γ irradiation, e.g., with 500 to 1000 cGy, or with 500, 1000, 2000, or 3000 cGy. Gamma irradiation of immune cells (e.g., NK-92 cells) at doses of between about 750 and 1000 Grays, e.g., 750, 800, 850, 900 and 950 Grays, is considered to be sufficient for this purpose. Additional forms of radiation, including, for example, ultraviolet radiation, may be employed. Suitable sources to use for this purpose include, for example, a 137C's source (Cis-US, Bedford, Mass.; Gammacell 40, Atomic Energy of Canada Ltd., Canada). Alternatively, the cells may include a suicide gene as described above.

In some embodiments, before immune cell (e.g., NK-92 cell)infusion, the subjects can be treated with a preparatory chemotherapy regimen, e.g., high cyclophosphamide and fludarabine (Hi-Cy [60 mg/kg×2 days]/Flu [25 mg/m²×5 days]), low cyclophosphamide (750 mg/m²) and methylprednisone (1000 mg/m²) or fludarabine alone (25 mg/m²×5 days), and or with total body irradiation, e.g., a dose of 200-500, e.g., 400 cGy, radiation.

### Combination Therapies: Checkpoint Inhibitors and Anti-Tumor Monoclonal mAbs

In some embodiments, the chimera-expressing immune cells (e.g., NK cells) capable of also expressing a pore-forming protein as described herein are administered as part of a therapeutic regimen that includes administration of one or more checkpoint blocking agents and/or anti-tumor antibodies. The NK cells can be administered concurrently, e.g., substantially simultaneously or sequentially, with the checkpoint blocking agents and/or anti-tumor antibodies, e.g., within 48, 24, 12, 6, 5, 4, 3, 2, or 1 hour, or within 45, 30, 20, or 15 minutes of administration of the checkpoint blocking agents and/or anti-tumor antibodies.

The term “antibody” as used berein refers to an immunoglobulin molecule or immunologically active portion thereof, i.e., an antigen-binding portion. Examples of immunologically active portions of immunoglobulin molecules include F(ab) and F(ab′)₂ fragments, which retain the ability to bind antigen. Such fragments can be obtained commercially, or using methods known in the art. For example, F(ab)2 fragments can be generated by treating the antibody with an enzyme such as pepsin, a non-specific endopeptidase that normally produces one F(ab)2 fragment and numerous small peptides of the Fc portion. The resulting F(ab)2 fragment is composed of two disulfide-connected Fab units. The Fc fragment is extensively degraded and can be separated from the F(ab)2 by dialysis, gel filtration or ion exchange chromatography. F(ab) fragments can be generated using papain, a non-specific thiol-endopeptidase that digests IgG molecules, in the presence of a reducing agent, into three fragments of similar size: two Fab fragments and one Fc fragment. When Fc fragments are of interest, papain is the enzyme of choice because it yields a 50.00 Dalton Fc fragment; to isolate the F(ab) fragments, the Fc fragments can be removed, e.g., by affinity purification using protein A/G. A number of kits are available commercially for generating F(ab) fragments, including the ImmunoPure IgG1 Fab and F(ab′)₂ Preparation Kit (Pierce Biotechnology, Rockford, IL). In addition, commercially available services for generating antigen-binding fragments can be used, e.g., Bio Express, West Lebanon, NH.

The antibody can be a polyclonal, monoclonal, recombinant, e.g., a chimeric, de-immunized or humanized, fully human, non-human, e.g., murine, or single chain antibody. In some embodiments the antibody has effector function and can fix complement. In some embodiments, the antibody has reduced or no ability to bind an Fc receptor. For example, the antibody can be an isotype or subtype, fragment or other mutant, which does not support binding to an Fc receptor, e.g., it has a mutagenized or deleted Fc receptor binding region. The antibody can be coupled to a toxin or imaging agent.

Therapeutic anti-tumor antibodies are also known in the art and include human, humanized and chimeric antibodies that bind to tumor antigens. The antibodies are typically monoclonal and can be, e.g., naked, conjugated, or bispecific. Specific examples include alemtuzumab, rituxumab, trastuzumab, ibritumomab, gemtuzumab, brentuximab, adotranstuzumab, blinatunomab, daratumumab and elotuzumab; abciximab; adalimumab; alefacept; basiliximab; belimumab; bezlotoxumab; canakinumab; certolizumab pegol; cetuximab; daclizumab; denosumab; efalizumab; elotuzumab; golimumab; inflectra; ipilimumab; ixekizumab; natalizumab; nivolumab; obinutuzumab; olaratumab; omalizumab; palivizumab; panitumumab; pembrolizumab; tocilizumab; secukinumab; and ustekinumab. A number of antibodies against cancer-related antigens are known; exemplary antibodies are described in Tables 2-3 (Ross et al., Am J Clin Pathol 119(4):472-485, 2003). The method can be used, e.g., to treat a subject who has a cancer that the anti-tumor antibody has been approved to treat (e.g., NK cells in combination with trastuzumab for a subject who has breast cancer, with bemtuximab in a subject who has Hodgkin lymphoma, with daratumumab in a subject who has multiple myeloma, or with elotuzumab in a subject who has multiple myeloma).

Checkpoint blocking agents are known in the art and include antibodies directed to CTLA-4 (e.g., ipilimumab, tremelimumab); PD-1 (e.g., nivolumab, pembrolizumab, BGB-A317); PD-L1 (e.g., atezolizumab, avelumab and durvalumab); CD40 (e.g., dacetuzumab, lucatumumab, bleselumab, tencliximab,); Tim3 (e.g., LY3321367, DCB-8, MBG453 and TSR-022); Lag3 (e.g., BMS-986016); and TIGIT (e.g., AB154; MK-7684; BMS-986207; ASP8374; Tiragolumab (MTIG7192A; RG6058); (Etigilimab (OMP-313M32)); 313R12).

Methods described herein encompass administering any of the chimera-expressing immune cells capable of also expressing a pore-forming protein described herein and an antibody directed to an immune checkpoint protein.

In some examples, methods include administering one or more types of chimera-expressing immune cells (e.g., administering CIRB21-expressing immune cells or administering CIRB-expressing immune cells and CIRB21-expressing immune cells) and one or more antibodies directed to an immune checkpoint protein (e.g., an anti-CTLA-4 antibody or an anti-PD-1 antibody and an anti-CTLA-4 antibody).

In some embodiments, methods described herein include administering CIRB-expressing immune cells capable of also expressing a holin and an antibody directed to an immune checkpoint protein (e.g., an anti-CTLA-4 antibody, an anti-PD-1 antibody, an anti-PD-L1 antibody, an anti-CD4-antibody, an anti-Tim3 antibody, an anti-Lag3 antibody, an anti-TIGIT antibody, or a combination thereof).

In some embodiments, methods described herein include administering CIRB21-expressing immune cells capable of also expressing a holin and an antibody directed to an immune checkpoint protein (e.g., an anti-CTLA-4 antibody, an anti-PD-1 antibody, an anti-PD-L1 antibody, an anti-CD4-antibody, an anti-Tim3 antibody, an anti-Lag3 antibody, an anti-TIGIT antibody, or a combination thereof).

In some embodiments, methods described herein include administering CIRB28-expressing immune cells capable of also expressing a holin and an antibody directed to an immune checkpoint protein (e.g., an anti-CTLA-4 antibody, an anti-PD-1 antibody, an anti-PD-L1 antibody, an anti-CD4-antibody, an anti-Tim3 antibody, an anti-Lag3 antibody, an anti-TIGIT antibody, or a combination thereof).

Exemplary anti-PD-1 antibodies that can be used in the methods described herein include those that bind to human PD-1; an exemplary PD-1 protein sequence is provided at NCBI Accession No. NP_005009.2. Exemplary antibodies are described in U.S. Pat. Nos. 8,008,449; 9,073,994; and U.S. 20110271358, including PF-06801591, AMP-224, BGB-A317, BI 754091, JS001, MEDI0680, PDR001, REGN2810, SHR-1210, TSR-042, pembrolizumab, nivolumab, avelumab, pidilizumab, and atezolizumab.

Exemplary anti-CD40 antibodies that can be used in the methods described herein include those that bind to human CD40; exemplary CD40 protein precursor sequences are provided at NCBI Accession No. NP_001241.1, NP_690593.1, NP_001309351.1, NP_001309350.1 and NP_001289682.1. Exemplary antibodies include those described in WO2002/088186; WO2007/124299; WO2011/123489; WO2012/149356; WO2012/111762; WO2014/070934; U.S. 20130011405; U.S. 20070148163; U.S. 20040120948; U.S. 20030165499; and U.S. Pat. No. 8,591,900, including dacetuzumab, lucatumumab, blesclumab, teneliximab. ADC-1013, CP-870,893, Chi Lob 7/4, HCD122, SGN-4, SEA-CD40, BMS-986004, and APX005M. In some embodiments, the anti-CD40 antibody is a CD40 agonist, and not a CD40 antagonist.

Exemplary CTLA-4 antibodies that can be used in the methods described herein include those that bind to human CTLA-4; exemplary CTLA-4 protein sequences are provided at NCBI Acc No. NP_005205.2. Exemplary antibodies include those described in Tarhini and Iqbal, Onco Targets Ther. 3:15-25 (2010); Storz, MAbs. 2016 January; 8(1): 10-26; U.S. 2009025274; U.S. Pat. Nos. 7,605,238; 6,984,720; EP1212422; U.S. Pat. Nos. 5,811,097; 5,855,887; 6,051,227; 6,682,736; EP1141028; and U.S. Pat. No. 7,741,345; and include ipilimumab, Tremelimumab, and EPR1476.

Exemplary anti-PD-L1 antibodies that can be used in the methods described herein include those that bind to human PD-L1; exemplary PD-L1 protein sequences are provided at NCBI Accession No. NP_001254635.1, NP_001300958.1, and NP_054862.1. Exemplary antibodies are described in U.S. 20170058033; WO2016/061142A1; WO2016/007235A1; WO2014/195852A1; and WO2013/079174A1, including BMS-936559 (MDX-1105), FAZ053, KN035, Atezolizumab (Tecentriq, MPDL3280A), Avelumab (Bavencio), and Durvalumab (Imfinzi, MEDI-4736).

Exemplary anti-Tim3 (also known as hepatitis A virus cellular receptor 2 or HAVCR2) antibodies that can be used in the methods described herein include those that bind to human Tim3; exemplary Tim3 sequences are provided at NCBI Accession No. NP_116171.3. Exemplary antibodies are described in WO2016071448; U.S. Pat. No. 8,552,156; and U.S. Pub. Nos. 20180298097; 20180251549; 20180230431; 20180072804; 20180016336; 20170313783; 20170114135; 20160257758; 20160257749; 20150086574; and 20130022623, and include LY3321367, DCB-8, MBG453 and TSR-022.

Exemplary anti-Lag3 antibodies that can be used in the methods described herein include those that bind to human Lag3; exemplary Lag3 sequences are provided at NCBI Accession No. NP_002277.4. Exemplary antibodies are described in Andrews et al., Immunol Rev. 2017 March; 276(1):80-96; Antoni et al., Am Soc Clin Oncol Educ Book. 2016; 35:e450-8; U.S. Pub. Nos. 20180326054; 20180251767; 20180230431; 20170334995; 20170290914; 20170101472; 20170022273; 20160303124, and include BMS-986016.

Exemplary anti-TIGIT antibodies that can be used in the methods described herein include those that bind to human TIGIT; an exemplary human TIGIT sequence is provided at NCBI Accession No. NP_776160.2. Exemplary antibodies include AB154; MK-7684; BMS-986207; ASP8374; Tiragolumab (MTIG7192A; RG6058); (Etigilimab (OMP-313M32)); 313R12. Sce, e.g., Harjunpää and Guillerey, Clin Exp Immunol 2019 Dec. 11; and U.S. Pub. Nos. 20200062859 and 20200040082

### EXAMPLES

The invention is further described in the following examples, which do not limit the scope of the invention described in the claims.

### Materials and Methods

The following materials and methods were used in the Examples below.

Chimera CIRB construction—IL2 cDNA was amplified from human brain total RNA by RTPCR using Forward primer 5′-TGCAGGATCCACTCACAGTAACCTCAACTCC-3′ (SEQ ID NO: 2) and reverse primer 5′-TGCACTCGAGAGTGAAACCATTTTAGAGCC-3′ (SEQ ID NO: 3) and cloned in BamHI-XhoI in pCDNA4-TO. To build the CIRB chimera we first constructed a chimera from IL2 and the extracellular domain of its receptor IL2Rα, which was amplified by RT-PCR from NK92 total RNA using forward oligo 5′-GGATTACCTTTTGTCAAAGCATCATCTCAACACTGACTGAGCAGAAGCTCATTTC GGAAGAAGACCTTGAAATGGAGACCAGTCAGTTTCCAGG-3′ (SEQ ID NO: 4), bridging IL2 C-terminal (12 amino acids before the stop codon), and contains the cMyc Tag, the sequence between amino acids 187-194 of IL2Rα as well as and the non-coding 3″ sequence of IL2 plasmid. This primer was used with reverse oligo 5′-CCTGATATGTTTTAAGTGGGAAGCACTTAATTATCAGATTGT TCTTCTACTCTTCCTCTGTCTCC-3′ (SEQ ID NO: 5). The amplified fragment was used, as an oligo to mutagenize IL2 wild type resulting in an IL2-IL2Rα chimera. To build CIRB final chimera construct, the IL2 receptor alpha chimera was used to amplify IL2 with a C-terminal cMyc tag followed by only the extra cellular domain of IL2Rα then followed by the N-terminal fragment of IL2Rβ using Forward 5′-TGCAGGATCCACTCACAGTAACCTCAACTCC-3′ (SEQ ID NO: 6) and reverse 5′-GGGAAGTGCCATTCACCGCGCAGGAAGTCTCACTCTCAGGA-3′ (SEQ ID NO: 7). This later introduces the N-terminal end of IL2Rβ. The product was then re-amplified using the same forward primer and reverse 5′-GGCTCTCGAGTTGTAG AAGCATGTGAACTGGGAAGTGCC ATTCACCGC-3″ (SEQ ID NO: 8). An XbaI site in IL2 was first removed by mutagenesis using primers forward 5′-CATCTTCAGTGCCTAGAAGAAGAACTC-3′ (SEQ ID NO: 9) and reverse 5′-GAGTTCTTCTTCTAGGCACTGAAGATG-3′ (SEQ ID NO: 10). IL2Rβ was then amplified using forward 5′-TTCCCAGTTCACATGCTTCTACAAGTCGA CAGCCAACATCTCCTG-3′ (SEQ ID NO: 11) and reverse 5′-AGCTTCTAGACTC GAGTTATCACACCAAGTGAGTTGGGTCCTGACCCTGG-3′ (SEQ ID NO: 12). Next the fragment IL2-cMye-IL2Rα was open Xho-XbaI and IL2Rβ was added as SalI-XbaI fragment to form the final chimera CIRB. Both IL2 and CIRB were transferred from pcDNA4-TO using SpeI (blunt end) and XhoI to CSCW-mcherry lentiviral vector digested with BamHI (blunt end) and XhoI. All constructs were sequenced and verified for Lentivirus integrity.

Lentivirus production and transduction—L-holin expressing lentiviral vector TLCV2 (1 μg) was transfected using 10 μl of Lipofectamine 2000 (cat #11668019) in 293T cells with 1 μg packaging vector pCMV-dR8.2 dvpr (Addgene Plasmid #8455) and pseudotyped with 1 μg of VSV using pCMV VSV plasmid.

iCasp9 retroviral vector pMSCV-F-del Casp9.IRES.GFP (addgene Plasmid #15567) was packaged similarly and using the same DNA ratios but using pCL-Eco (addgene Plasmid #12371) instead of pCMV-dR8.2 dvpr.

Viral particles were collected 3 days post transfection and used to infect GL261 or NK92 cell lines expressing the chimera CIRB21 (IL2-IL2RB-IL21R).

GL261 clonal selection—Prior to cloning L-Holin, GL261 were selected by puromycin, while icasp9 GL261 cells were sorted by flow cytometry to select GFP expressing cells.

L-Holin GL261 and icasp9 GL261 clones were selected as single cells by extreme dilution in 96 well plates. Clones were grown and evaluated for their response to dox (L-Holin) or AP1093 (icasp9).

NK92^{CIRB21} pool selection—NK92^{CIRB21} cells expressing L-Holin were selected with puromycin and NK92^{CIRB21} cells expressing icasp9 and GFP were sorted by flow cytometry for GFP.

L-Holin killing assays—Cells selected cell for L-Holin were tested in a 24 well plate using 32,000 cells per well. 24 hours later doxycycline was added at 1 μg/ml for 4-5 days. Cells selected for icasp9 were tested in a 24 well plate using 32,000 cells per well. 24 hours later dimerizer drug AP1093 was added at 2 or 10 nM for 4-5 days. Surviving cells were quantified using crustal violet assay as described in (Jounaidi et al, Cancer Research 2017).

Cytotoxic activity of NK92^{CIRB21} cells—32×10³ PC-3 cancer cells were first plated in a 24-well plate for either 24 hours prior to adding NK92^{CIRB21} or NK92^{CIRB21+Holin}. Co-cultured cells were then incubated for 4 days. Cell viability of cancer cells after this time was determined using a 0.1% crystal violet in a 10% alcohol solution followed by extraction using 70% ethanol and reading absorbance at 595 nm.

Survival of GL261 and NK92 cells—Viability of GL261 and NK92 cells expressing icasp9 or L-holin was determined using Trypan Blue.

Statistical Analysis—Statistical significance of differences was determined by two-tailed Student's test, a one-way ANOVA, paired Tukey's Multiple Comparison test. All tests included comparisons to untreated samples or as indicated in the text. Statistical significance is indicated by *P<0.05, **P<0.01, ***P<0.001, ****P<0.001. Analyses were performed using Prism software version 6 (GraphPad Software).

### Example 1. Design and Construction of the CIRB Chimera

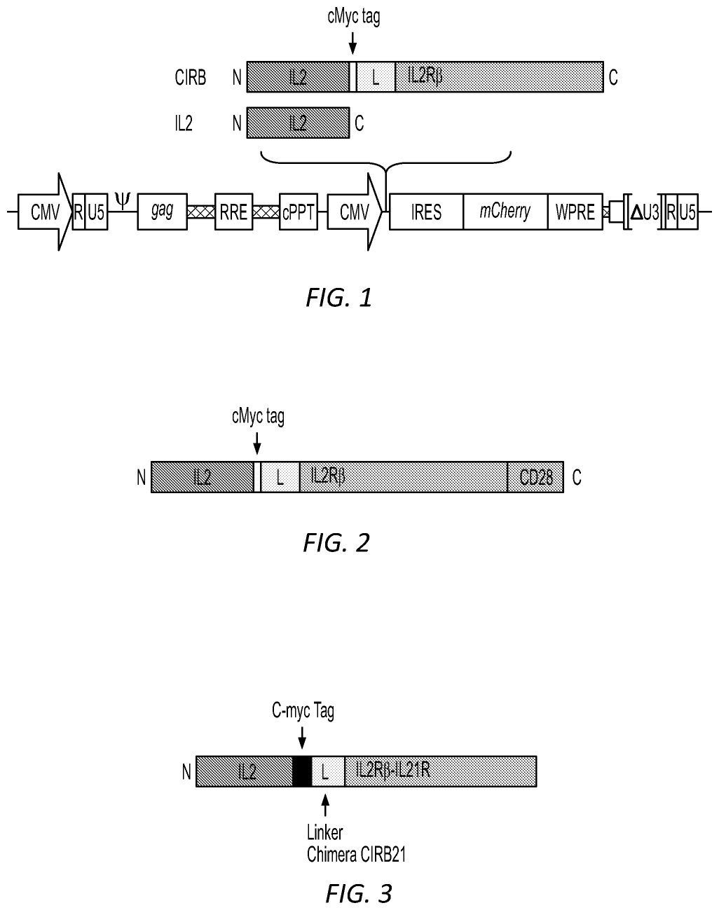

The quaternary crystal structure of IL2 and its receptors complex (20) shows that the C-terminal end of IL2 and the N-terminal residue of IL2Rβ are separated by 41 Å. For a linker between IL2 and the N-terminus of IL2Rβ we choose the extracellular domain of IL2Rα (EMETSQFPGEEKPQASPEGRPESETSC (SEQ ID NO: 28)). A cMyc tag (EQKLISEEDL (SEQ ID NO: 29)) was added between IL2 and the linker. The fully mature receptor IL2Rβ protein coding sequence (without signal peptide) was placed after the linker to yield the full chimera CIRB (FIG. 1). Both CIRB and IL2 were cloned in a lentiviral vector co-expressing mCherry. The linker fold was predicted computationally to be a helix-dominated structure. Linker flexibility was assessed using the computational method of Karplus and Shultz method (47) which, indicates better than average flexibility (1 or greater on a 0 to 2 scale) at all the peptide linkages.

The resulting sequences are shown below.

Nucleotide Sequence of IL2-IL2Rβ:
(SEQ ID NO: 30)
ATGTACAGGATGCAACTCCTGTCTTGCATTGCACTAAGTCTTGCACTTGTCACAAACAGTGC
ACCTACTTCAAGTTCTACAAAGAAAACACAGCTACAACTGGAGCATTTACTGCTGGATTTAC
AGATGATTTTGAATGGAATTAATAATTACAAGAATCCCAAACTCACCAGGATGCTCACATTT
AAGTTTTACATGCCCAAGAAGGCCACAGAACTGAAACATCTTCAGTGCCTAGAAGAAGAACT
CAAACCTCTGGAGGAAGTGCTAAATTTAGCTCAAAGCAAAAACTTTCACTTAAGACCCAGGG
ACTTAATCAGCAATATCAACGTAATAGTTCTGGAACTAAAGGGATCTGAAACAACATTCATG
TGTGAATATGCTGATGAGACAGCAACCATTGTAGAATTTCTGAACAGATGGATTACCTTTTG
TCAAAGCATCATCTCAACACTGACTGAGCAGAAGCTCATTTCGGAAGAAGACCTTGAAATGG
AGACCAGTCAGTTTCCAGGTGAAGAGAAGCCTCAGGCAAGCCCCGAAGGCCGTCCTGAGAGT
GAGACTTCCTGCGCGGTGAATGGCACTTCCCAGTTCACATGCTTCTACAACTCGCGAGCCAA
CATCTCCTGTGTCTGGAGCCAAGATGGGGCTCTGCAGGACACTTCCTGCCAAGTCCATGCCT
GGCCGGACAGACGGCGGTGGAACCAAACCTGTGAGCTGCTCCCCGTGAGTCAAGCATCCTGG
GCCTGCAACCTGATCCTCGGAGCCCCAGATTCTCAGAAACTGACCACAGTTGACATCGTCAC
CCTGAGGGTGCTGTGTCGTGAGGGGGTGCGATGGAGGGTGATGGCCATCCAGGACTTCAAGC
CCTTTGAGAACCTTCGCCTGATGGCCCCCATCTCCCTCCAAGTTGTCCACGTGGAGACCCAC
AGATGCAACATAAGCTGGGAAATCTCCCAAGCCTCCCACTACTTTGAAAGACACCTGGAGTT
CGAGGCCCGGACGCTGTCCCCAGGCCACACCTGGGAGGAGGCCCCCCTGCTGACTCTCAAGC
AGAAGCAGGAATGGATCTGCCTGGAGACGCTCACCCCAGACACCCAGTATGAGTTTCAGGTG
CGGGTCAAGCCTCTGCAAGGCGAGTTCACGACCTGGAGCCCCTGGAGCCAGCCCCTGGCCTT
CAGGACAAAGCCTGCAGCCCTTGGGAAGGACACCATTCCGTGGCTCGGCCACCTCCTCGTGG
GTCTCAGCGGGGCTTTTGGCTTCATCATCTTAGTGTACTTGCTGATCAACTGCAGGAACACC
GGGCCATGGCTGAAGAAGGTCCTGAAGTGTAACACCCCAGACCCCTCGAAGTTCTTTTCCCA
GCTGAGCTCAGAGCATGGAGGAGACGTCCAGAAGTGGCTCTCTTCGCCCTTCCCCTCATCGT
CCTTCAGCCCTGGCGGCCTGGCACCTGAGATCTCGCCACTAGAAGTGCTGGAGAGGGACAAG
GTGACGCAGCTGCTCCTGCAGCAGGACAAGGTGCCTGAGCCCGCATCCTTAAGCAGCAACCA
CTCGCTGACCAGCTGCTTCACCAACCAGGGTTACTTCTTCTTCCACCTCCCGGATGCCTTGG
AGATAGAGGCCTGCCAGGTGTACTTTACTTACGACCCCTACTCAGAGGAAGACCCTGATGAG
GGTGTGGCCGGGGCACCCACAGGGTCTTCCCCCCAACCCCTGCAGCCTCTGTCAGGGGAGGA
CGACGCCTACTGCACCTTCCCCTCCAGGGATGACCTGCTGCTCTTCTCCCCCAGTCTCCTCG
GTGGCCCCAGCCCCCCAAGCACTGCCCCTGGGGGCAGTGGGGCCGGTGAAGAGAGGATGCCC
CCTTCTTTGCAAGAAAGAGTCCCCAGAGACTGGGACCCCCAGCCCCTGGGGCCTCCCACCCC
AGGAGTCCCAGACCTGGTGGATTTTCAGCCACCCCCTGAGCTGGTGCTGCGAGAGGCTGGGG
AGGAGGTCCCTGACGCTGGCCCCAGGGAGGGAGTCAGTTTCCCCTGGTCCAGGCCTCCTGGG
CAGGGGGAGTTCAGGGCCCTTAATGCTCGCCTGCCCCTGAACACTGATGCCTACTTGTCCCT
CCAAGAACTCCAGGGTCAGGACCCAACTCACTTGGTGTGA
Protein Sequence of IL2-IL2Rβ
(SEQ ID NO: 31)
MYRMQLLSCIALSLALVTNSAPTSSSTKKTQLQLEHLLLDLQMILNGINNYKNPKLTRMLTF
KFYMPKKATELKHLQCLEEELKPLEEVLNLAQSKNFHLRPRDLISNINVIVLELKGSETTFM
CEYADETATIVEFLNRWITFCQSIISTLTEQKLISEEDLEMETSQFPGEEKPQASPEGRPES
ETSCAVNGTSQFTCFYNSRANISCVWSQDGALQDTSCQVHAWPDRRRWNQTCELLPVSQASW
ACNLILGAPDSQKLTTVDIVTLRVLCREGVRWRVMAIQDFKPFENLRLMAPISLQVVHVETH
RCNISWEISQASHYFERHLEFEARTLSPGHTWEEAPLLTLKQKQEWICLETLTPDTQYEFQV
RVKPLQGEFTTWSPWSQPLAFRTKPAALGKDTIPWLGHLLVGLSGAFGFIILVYLLINCRNT
GPWLKKVLKCNTPDPSKFFSQLSSEHGGDVQKWLSSPFPSSSFSPGGLAPEISPLEVLERDK
VTQLLLQQDKVPEPASLSSNHSLTSCFTNQGYFFFHLPDALEIEACQVYFTYDPYSEEDPDE
GVAGAPTGSSPQPLQPLSGEDDAYCTFPSRDDLLLFSPSLLGGPSPPSTAPGGSGAGEERMP
PSLQERVPRDWDPQPLGPPTPGVPDLVDFQPPPELVLREAGEEVPDAGPREGVSFPWSRPPG
QGEFRALNARLPLNTDAYLSLQELQGQDPTHLV*

### Example 2. Design and Construction of a CIRB21 Chimera

Interleukins ILA, IL7, IL9, IL15 and IL21 belong to the same family as IL2, and use the same common IL2Rg. They all have their own private receptors, except for IL2 and IL15, which use IL2Rβ in addition to their own alpha receptors. In this Example, the entire cytoplasmic domain of IL21R was cloned then added Head-to-Tail to the C-terminal of IL2Rβ in the chimera CIRB. This resulted in a novel IL2-IL2Rβ-IL21R chimera (called CIRB21, exemplified in FIG. 3), which was then introduced in NK92 cells to yield NK92^{CIRB21}.

Nucleotide Sequence of IL2-IL2Rβ-IL21R (CIRB21)
(SEQ ID NO: 32)
ATGTACAGGATGCAACTCCTGTCTTGCATTGCACTAAGTCTTGCACTTGTCACAAACAGTGC
ACCTACTTCAAGTTCTACAAAGAAAACACAGCTACAACTGGAGCATTTACTGCTGGATTTAC
AGATGATTTTGAATGGAATTAATAATTACAAGAATCCCAAACTCACCAGGATGCTCACATTT
AAGTTTTACATGCCCAAGAAGGCCACAGAACTGAAACATCTTCAGTGCCTAGAAGAAGAACT
CAAACCTCTGGAGGAAGTGCTAAATTTAGCTCAAAGCAAAAACTTTCACTTAAGACCCAGGG
ACTTAATCAGCAATATCAACGTAATAGTTCTGGAACTAAAGGGATCTGAAACAACATTCATG
TGTGAATATGCTGATGAGACAGCAACCATTGTAGAATTTCTGAACAGATGGATTACCTTTTG
TCAAAGCATCATCTCAACACTGACTGAGCAGAAGCTCATTTCGGAAGAAGACCTTGAAATGG
AGACCAGTCAGTTTCCAGGTGAAGAGAAGCCTCAGGCAAGCCCCGAAGGCCGTCCTGAGAGT
GAGACTTCCTGCGCGGTGAATGGCACTTCCCAGTTCACATGCTTCTACAACTCGCGAGCCAA
CATCTCCTGTGTCTGGAGCCAAGATGGGGCTCTGCAGGACACTTCCTGCCAAGTCCATGCCT
GGCCGGACAGACGGCGGTGGAACCAAACCTGTGAGCTGCTCCCCGTGAGTCAAGCATCCTGG
GCCTGCAACCTGATCCTCGGAGCCCCAGATTCTCAGAAACTGACCACAGTTGACATCGTCAC
CCTGAGGGTGCTGTGTCGTGAGGGGGTGCGATGGAGGGTGATGGCCATCCAGGACTTCAAGC
CCTTTGAGAACCTTCGCCTGATGGCCCCCATCTCCCTCCAAGTTGTCCACGTGGAGACCCAC
AGATGCAACATAAGCTGGGAAATCTCCCAAGCCTCCCACTACTTTGAAAGACACCTGGAGTT
CGAGGCCCGGACGCTGTCCCCAGGCCACACCTGGGAGGAGGCCCCCCTGCTGACTCTCAAGC
AGAAGCAGGAATGGATCTGCCTGGAGACGCTCACCCCAGACACCCAGTATGAGTTTCAGGTG
CGGGTCAAGCCTCTGCAAGGCGAGTTCACGACCTGGAGCCCCTGGAGCCAGCCCCTGGCCTT
CAGGACAAAGCCTGCAGCCCTTGGGAAGGACACCATTCCGTGGCTCGGCCACCTCCTCGTGG
GTCTCAGCGGGGCTTTTGGCTTCATCATCTTAGTGTACTTGCTGATCAACTGCAGGAACACC
GGGCCATGGCTGAAGAAGGTCCTGAAGTGTAACACCCCAGACCCCTCGAAGTTCTTTTCCCA
GCTGAGCTCAGAGCATGGAGGAGACGTCCAGAAGTGGCTCTCTTCGCCCTTCCCCTCATCGT
CCTTCAGCCCTGGCGGCCTGGCACCTGAGATCTCGCCACTAGAAGTGCTGGAGAGGGACAAG
GTGACGCAGCTGCTCCTGCAGCAGGACAAGGTGCCTGAGCCCGCATCCTTAAGCAGCAACCA
CTCGCTGACCAGCTGCTTCACCAACCAGGGTTACTTCTTCTTCCACCTCCCGGATGCCTTGG
AGATAGAGGCCTGCCAGGTGTACTTTACTTACGACCCCTACTCAGAGGAAGACCCTGATGAG
GGTGTGGCCGGGGCACCCACAGGGTCTTCCCCCCAACCCCTGCAGCCTCTGTCAGGGGAGGA
CGACGCCTACTGCACCTTCCCCTCCAGGGATGACCTGCTGCTCTTCTCCCCCAGTCTCCTCG
GTGGCCCCAGCCCCCCAAGCACTGCCCCTGGGGGCAGTGGGGCCGGTGAAGAGAGGATGCCC
CCTTCTTTGCAAGAAAGAGTCCCCAGAGACTGGGACCCCCAGCCCCTGGGGCCTCCCACCCC
AGGAGTCCCAGACCTGGTGGATTTTCAGCCACCCCCTGAGCTGGTGCTGCGAGAGGCTGGGG
AGGAGGTCCCTGACGCTGGCCCCAGGGAGGGAGTCAGTTTCCCCTGGTCCAGGCCTCCTGGG
CAGGGGGAGTTCAGGGCCCTTAATGCTCGCCTGCCCCTGAACACTGATGCCTACTTGTCCCT
CCAAGAACTCCAGGGTCAGGACCCAACTCACTTGGTGAGCCTGAAGACCCATCCATTGTGGA
GGCTATGGAAGAAGATATGGGCCGTCCCCAGCCCTGAGCGGTTCTTCATGCCCCTGTACAAG
GGCTGCAGCGGAGACTTCAAGAAATGGGTGGGTGCACCCTTCACTGGCTCCAGCCTGGAGCT
GGGACCCTGGAGCCCAGAGGTGCCCTCCACCCTGGAGGTGTACAGCTGCCACCCACCACGGA
GCCCGGCCAAGAGGCTGCAGCTCACGGAGCTACAAGAACCAGCAGAGCTGGTGGAGTCTGAC
GGTGTGCCCAAGCCCAGCTTCTGGCCGACAGCCCAGAACTCGGGGGGCTCAGCTTACAGTGA
GGAGAGGGATCGGCCATACGGCCTGGTGTCCATTGACACAGTGACTGTGCTAGATGCAGAGG
GGCCATGCACCTGGCCCTGCAGCTGTGAGGATGACGGCTACCCAGCCCTGGACCTGGATGCT
GGCCTGGAGCCCAGCCCAGGCCTAGAGGACCCACTCTTGGATGCAGGGACCACAGTCCTGTC
CTGTGGCTGTGTCTCAGCTGGCAGCCCTGGGCTAGGAGGGCCCCTGGGAAGCCTCCTGGACA
GACTAAAGCCACCCCTTGCAGATGGGGAGGACTGGGCTGGGGGACTGCCCTGGGGTGGCCGG
TCACCTGGAGGGGTCTCAGAGAGTGAGGCGGGCTCACCCCTGGCCGGCCTGGATATGGACAC
GTTTGACAGTGGCTTTGTGGGCTCTGACTGCAGCAGCCCTGTGGAGTGTGACTTCACCAGCC
CCGGGGACGAAGGACCCCCCCGGAGCTACCTCCGCCAGTGGGTGGTCATTCCTCCGCCACTT
TCGAGCCCTGGACCCCAGGCCAGCTAA
Protein Sequence of IL2-IL2Rβ-IL21R (CIRB21)
(SEQ ID NO: 33)
MYRMQLLSCIALSLALVTNSAPTSSSTKKTQLQLEHLLLDLQMILNGINNYKNPKLTRMLTF
KFYMPKKATELKHLQCLEEELKPLEEVLNLAQSKNFHLRPRDLISNINVIVLELKGSETTFM
CEYADETATIVEFLNRWITFCQSIISTLTEQKLISEEDLEMETSQFPGEEKPQASPEGRPES
ETSCAVNGTSQFTCFYNSRANISCVWSQDGALQDTSCQVHAWPDRRRWNQTCELLPVSQASW
ACNLILGAPDSQKLTTVDIVTLRVLCREGVRWRVMAIQDFKPFENLRLMAPISLQVVHVETH
RCNISWEISQASHYFERHLEFEARTLSPGHTWEEAPLLTLKQKQEWICLETLTPDTQYEFQV
RVKPLQGEFTTWSPWSQPLAFRTKPAALGKDTIPWLGHLLVGLSGAFGFIILVYLLINCRNT
GPWLKKVLKCNTPDPSKFFSQLSSEHGGDVQKWLSSPFPSSSFSPGGLAPEISPLEVLERDK
VTQLLLQQDKVPEPASLSSNHSLTSCFTNQGYFFFHLPDALEIEACQVYFTYDPYSEEDPDE
GVAGAPTGSSPQPLQPLSGEDDAYCTFPSRDDLLLFSPSLLGGPSPPSTAPGGSGAGEERMP
PSLQERVPRDWDPQPLGPPTPGVPDLVDFQPPPELVLREAGEEVPDAGPREGVSFPWSRPPG
QGEFRALNARLPLNTDAYLSLQELQGQDPTHLVSLKTHPLWRLWKKIWAVPSPERFFMPLYK
GCSGDFKKWVGAPFTGSSLELGPWSPEVPSTLEVYSCHPPRSPAKRLQLTELQEPAELVESD
GVPKPSFWPTAQNSGGSAYSEERDRPYGLVSIDTVTVLDAEGPCTWPCSCEDDGYPALDLDA
GLEPSPGLEDPLLDAGTTVLSCGCVSAGSPGLGGPLGSLLDRLKPPLADGEDWAGGLPWGGR
SPGGVSESEAGSPLAGLDMDTFDSGFVGSDCSSPVECDFTSPGDEGPPRSYLRQWVVIPPPL
SSPGPQAS*

### Example 3. Design and Construction of the CIRB, CIRB21, CIRB28 Chimeras or CAR and L-Holin Expression Cassette

Lambda Holin (L-Holin) gene sequence from KM823530.1 was used to create an optimized version of L-Holin for improved expression in mammals. The optimized sequence created is shown below:

(SEQ ID NO: 13)
ATGCCGGAAAAACACGATCTGCTGGCGGCGATTCTGGCGGCGAAGGAAC
AGGGTATTGGCGCGATTCTGGCGTTTGCGATGGCGTACCTGCGTGGTCG
TTATAACGGTGGCGCGTTCACCAAGACCGTGATCGACGCGACCATGTGC
GCGATCATTGCGTGGTTCATTCGTGACCTGCTGGATTTTGCGGGTCTGA
GCAGCAACCTGGCGTACATCACCAGCGTTTTTATCGGTTATATTGGCAC
CGATAGCATTGGCAGCCTGATTAAGCGTTTTGCGGCGAAGAAAGCGGGT
GTGGAAGACGGTCGTAACCAATAA

The L-Holin was cloned in the TLCV2 plasmid from Addgene (Plasmid #87360), between the AgeI and BamHI restriction sites using a forward primer (L-Holin sense AgeI; GAGAATACCGGTTGCGCTGCCACCATGCCGGAAAAACAC (SEQ ID NO: 14)) and a reverse primer (L-Holin reverse BamHI; AGACGGTCGTAACCAAGGATCCGGAGAG (SEQ ID NO: 15)).

L-Holin cloned between AgeI and BamHI in the TLCV2 plasmid is in tandem with GFP with a T2A peptide between them. Both are under the control of a tight tetracycline (TRE) regulated promoter. The puromycin and the repressor rTTA are under the control of the EF-1 core promoter.

Additional activating genes such as CARs, CIRB or CIRB21 or other activating cassettes with their promoters and P2A joining sequences could be cloned in the same vector between NheI and BsiWI.

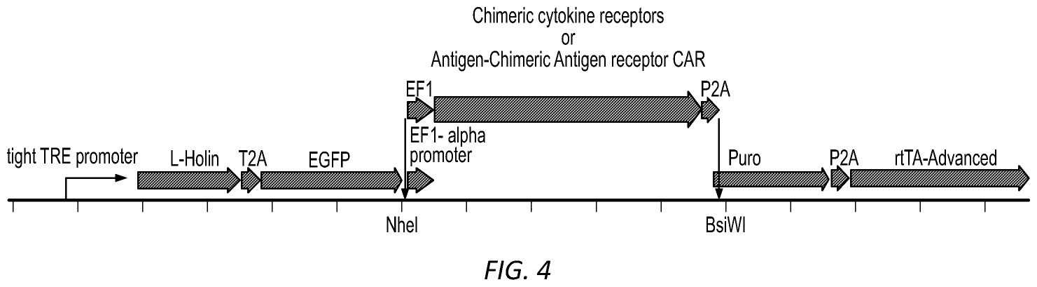

A schematic depiction of the expression cassette described herein is shown in FIG. 4. An example of a nucleic acid sequence of the expression cassette is shown below. The start of each component of the expression cassette is noted with dashes and parenthesis.

(expression cassette; SEQ ID NO: 16)
---AATTCACTTTGGCCGCGAATCGATATGTC---
(TRE promoter; SEQ ID NO: 17)
--GAGTTTACTCCCTATCAGTGATAGAGAACGTATGTCGAGTTTACTCCCTATCAGTGA
TAGAGAACGATGTCGAGTTTACTCCCTATCAGTGATAGAGAACGTATGTCGAGTTTA
CTCCCTATCAGTGATAGAGAACGTATGTCGAGTTTACTCCCTATCAGTGATAGAGAA
CGTATGTCGAGTTTATCCCTATCAGTGATAGAGAACGTATGTCGAGTTTACTCCCTA
TCAGTGATAGAGAACGTATGTCGAGGTAGGCGTGTACGGTGGGAGGCCTATATAAGC
AGAGCTCGTTTAGTGAACCGTCAGATCGCCTGGAGAATACCGGTTGCGCTGCCACC---
(L-Holin; SEQ ID NO: 18)
--ATGCCGGAAAAACACGATCTGCTGGCGGCGATTCTGGCGGCGAAGGAACAGGGTATT
GGCGCGATTCTGGCGTTTGCGATGGCGTACCTGCGTGGTCGTTATAACGGTGGCGCG
TTCACCAAGACCGTGATCGACGCGACCATGTGCGCGATCATTGCGTGGTTCATTCGT
GACCTGCTGGATTTTGGGGGTCTGAGCAGCAACCTGGCGTACATCACCAGCGTTTTT
ATCGGTTATATTGGCACCGATAGCATTGGCAGCCTGATTAAGCGTTTTGCGGCGAAG
AAAGCGGGTGTGGAAGACGGTCGTAACCAAGGATCCGGA---
(T2A; SEQ ID NO: 19)
--GAGGGCAGAGGAAGTCTGCTAACATGCGGTGACGTCGAGGAGAATCCTGGCCCA--
(GFP; SEQ ID NO: 20)
-GTGAGCAAGGGCGAGGAGCTGTTCACCGGGGTGGTGCCCATCCTGGTCGAGCTGGAC
GGCGACGTAAACGGCCACAAGTTCAGCGTGTCCGGCGAGGGCGAGGGCGATGCCACC
TACGGCAAGCTGACCCTGAAGTTCATCTGCACCACCGGCAAGCTGCCCGTGCCCTGG
CCCACCCTCGTGACCACCCTGACCTACGGCGTGCAGTGCTTCAGCCGCTACCCCGAC
CACATGAAGCAGCACGACTTCTTCAAGTCCGCCATGCCCGAAGGCTACGTCCAGGAG
CGCACCATCTTCTTCAAGGACGACGGCAACTACAAGACCCGCGCCGAGGTGAAGTTC
GAGGGCGACACCCTGGTGAACCGCATCGAGCTGAAGGGCATCGACTTCAAGGAGGAC
GGCAACATCCTGGGGCACAAGCTGGAGTACAACTACAACAGCCACAACGTCTATATC
ATGGCCGACAAGCAGAAGAACGGCATCAAGGTGAACTTCAAGATCCGCCACAACATC
GAGGACGGCAGCGTGCAGCTCGCCGACCACTACCAGCAGAACACCCCCATCGGCGAC
GGCCCCGTGCTGCTGCCCGACAACCACTACCTGAGCACCCAGTCCGCCCTGAGCAAA
GACCCCAACGAGAAGCGCGATCACATGGTCCTGCTGGAGTTCGTGACCGCCGCCGGG
ATCACTCTCGGCATGGACGAGCTGTACAAGTAA--
(NheI; SEQ ID NO: 21)
-GCTAGCATCC---
(EF-1; SEQ ID NO: 22)
-GGGCAGAGCGCACATCGCCCACAGTCCCCGAGAAGTTGGGGGGAGGGGTCGGCAATT
GATCCGGTGCCTAGAGAAGGTGGCGCGGGGTAAACTGGGAAAGTGATGTCGTGTACT
GGCTCCGCCTTTTTCCCGAGGGGGGGGAGAACCGTATATAAGTGCAGTAGTCGCCG
TGAACGTTCTTTTTCGCAACGGGTTTGCCGCCAGAACACAGACTGCGATCGCAATGT
ACAGT----
(Puro; SEQ ID NO: 23)
--ATGACCGAGTACAAGCCCACGGTGCGCCTCGCCACCCGCGACGACGTCCCCAG---
(BsiWI)----GGCCGTA---
(Puro; SEQ ID NO: 24)
-CGCACCCTCGCCGCCGCGTTCGCCGACTACCCCGCCACGCGCCACACCGTCGATCCG
GACCGCCACATCGAGCGGGTCACCGAGCTGCAAGAACTCTTCCTCACGCGCGTCGGG
CTCGACATCGGCAAGGTGTGGGTCGCGGACGACGGCGCCGCGGTGGCGGTCTGGACC
ACGCCGGAGAGCGTCGAAGCGGGGGCGGTGTTCGCCGAGATCGGCCCGCGCATGGCC
GAGTTGAGCGGTTCCCGGCTGGCCGCGCAGCAACAGATGGAAGGCCTCCTGGCGCCG
CACCGGCCCAAGGAGCCCGCGTGGTTCCTGGCCACCGTCGGAGTCTCGCCCGACCAC
CAGGGCAAGGGTCTGGGCAGCGCCGTCGTGCTCCCCGGAGTGGAGGCGGCCGAGCGC
GCCGGGGTGCCCGCCTTCCTGGAGACCTCCGCGCCCCGCAACCTCCCCTTCTACGAG
CGGCTCGGCTTCACCGTCACCGCCGACGTCGAGGTGCCCGAAGGACCGCGCACCTGG
TGCATGACCCGCAAGCCCGGTGCCGGTTCCGGC----
(P2A; SEQ ID NO: 25)
---GCAACAAACTTCTCTCTGCTGAAACAAGCCGGAGATGTCGAAGAGAATCCTGGACCG---
(rTTA; SEQ ID NO: 26)
---ATGTCTAGACTGGACAAGAGCAAAGTCATAAACGGAGCTCTGGAATTACTCAATGGT
GTCGGTATCGAAGGCCTGACGACAAGGAAACTCGCTCAAAAGCTGGGAGTTGAGCAG
CCTACCCTGTACTGGCACGTGAAGAACAAGCGGGCCCTGCTCGATGCCCTGCCAATC
GAGATGCTGGACAGGCATCATACCCACTTCTGCCCCCTGGAAGGCGAGTCATGGCAA
GACTTTCTGCGGAACAACGCCAAGTCATACCGCTGTGCTCTCCTCTCACATCGCGAC
GGGGCTAAAGTGCATCTCGGCACCCGCCCAACAGAGAAACAGTACGAAACCCTGGAA
AATCAGCTCGCGTTCCTGTGTCAGCAAGGCTTCTCCCTGGAGAACGCACTGTACGCT
CTGTCCGCCGTGGGCCACTTTACACTGGGCTGCGTATTGGAGGAACAGGAGCATCAA
GTAGCAAAAGAGGAAAGAGAGACACCTACCACCGATTCTATGCCCCCACTTCTGAGA
CAAGCAATTGAGCTGTTCGACCGGCAGGGAGCCGAACCTGCCTTCCTTTTCGGCCTG
GAACTAATCATATGTGGCCTGGAGAAACAGCTAAAGTGCGAAAGCGGCGGGCCGACC
GACGCCCTTGACGATTTTGACTTAGACATGCTCCCAGCCGATGCCCTTGACGACTTT
GACCTTGATATGCTGCCTGCTGACGCTCTTGACGATTTTGACCTTGACATGCTCCCC
GGGTAA

### Example 4. L-Holin Expression Triggers Death in Proliferating Cells

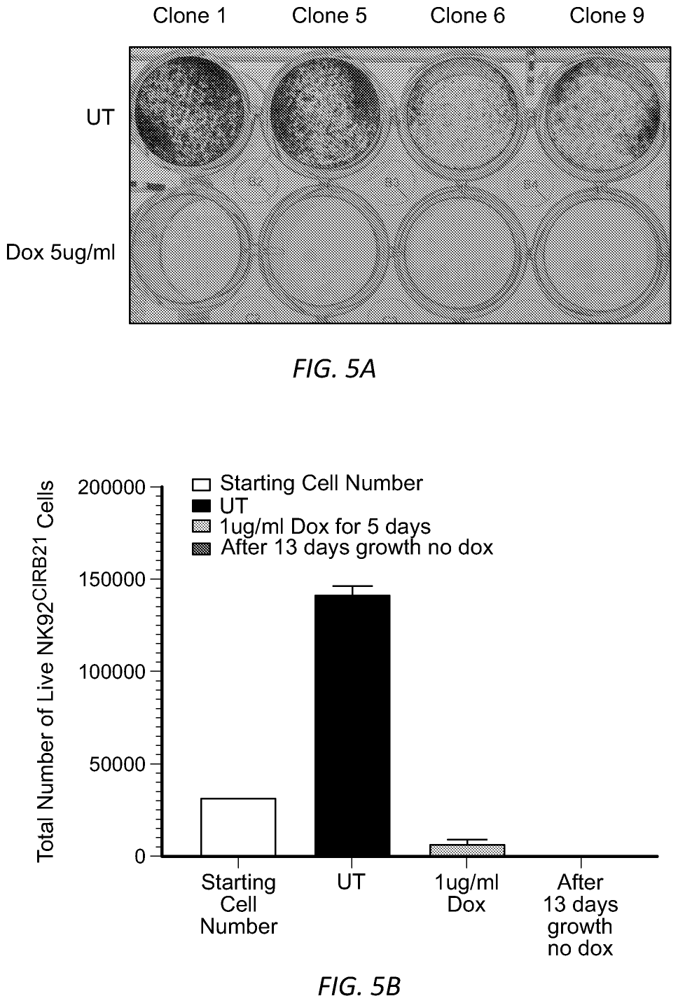

Mouse glioblastoma cell line GL261 was infected by a retrovirus expressing L-Holin. Clones were selected, L-Holin expression was induced with doxycycline, and cell viability was determined. Four clones were tested: 2 clones with high expression levels of L-Holin (Clone 1, Clone 5) and 2 clones with low expression levels of L-Holin (Clone 6, Clone 9). The majority, if not all, of GL261 cells died as a result of L-Holin expression including cells expressing L-Holin at low levels (FIG. 5A). Similar results were obtained in human NK92^{CIRB21} cells infected with a lentivirus expressing L-Holin (FIG. 5B). These results demonstrate that expression of L-Holin, even at low levels, in mammalian cells results in cell death.

### Example 5. L-Holin Does not Interfere with Cytotoxicity of NK92 Cells

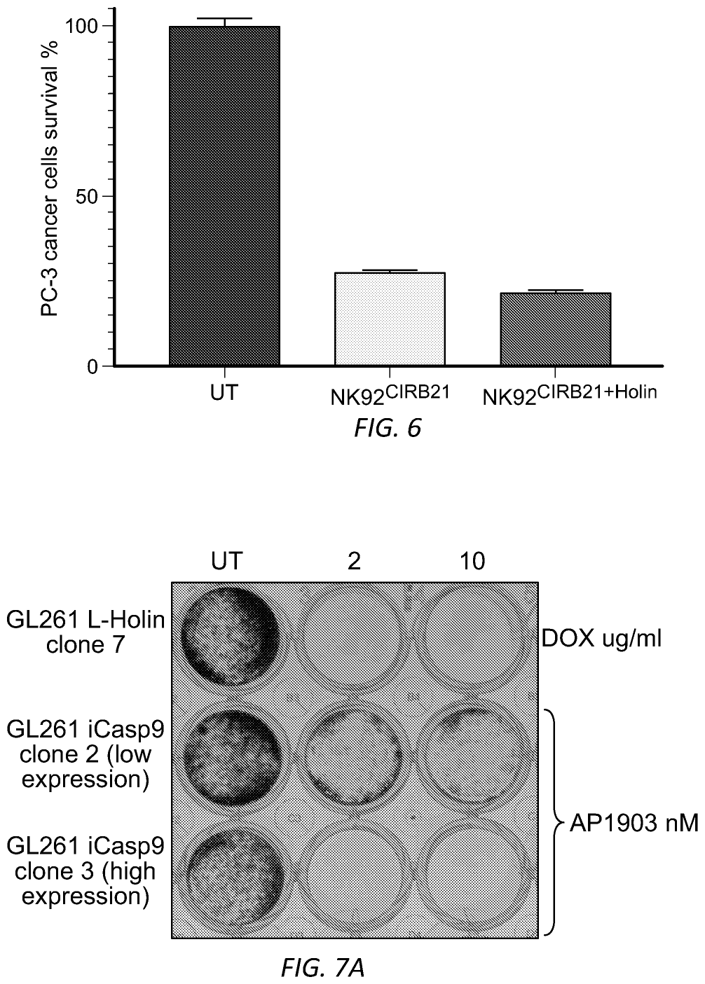

Previous studies demonstrated that NK92^{CIRB} cells are cytotoxic against cancer cells. See, e.g., Example 3 of U.S. 2020/0316118, incorporated herein by reference in its entirety. In this Example, the cytotoxicity of NK92^{CIRB21} cells capable of expressing L-Holin (NK92^{CIRB21+Holin} cells) were compared with non-holin expressing NK92^{CIRB21} cells. Experiments were also performed with untransduced cells as a control. As shown in FIG. 6, the cytotoxicity against PC-3 cancer cells of NK92^{CIRB21+Holin} cells was similar to that of NK92^{CIRB21} cells. These results demonstrate that the TET-ON system controls expression of L-Holin and does not interfere with the cytotoxicity of NK92^{CIRB} cells against cancer cells.

### Example 6. L-Holin Expression Triggers Cell Death More Efficiently Than icasp9

We compared the ability of L-Holin to trigger cell death to that of icasp9 in GL261 cells. GL261 cells were infected by a retrovirus expressing L-Holin or a retrovirus expressing icasp9. Clones were selected, L-Holin expression was induced with doxycycline, and cell viability was determined. Two icasp9 expressing clones were tested: 1 clone with high expression levels of icasp9 (Clone 3) and 1 clone with low expression levels of icasp9 (Clone 2). icasp9 cells were treated with either 2 nM or 10 nM of AP1903.

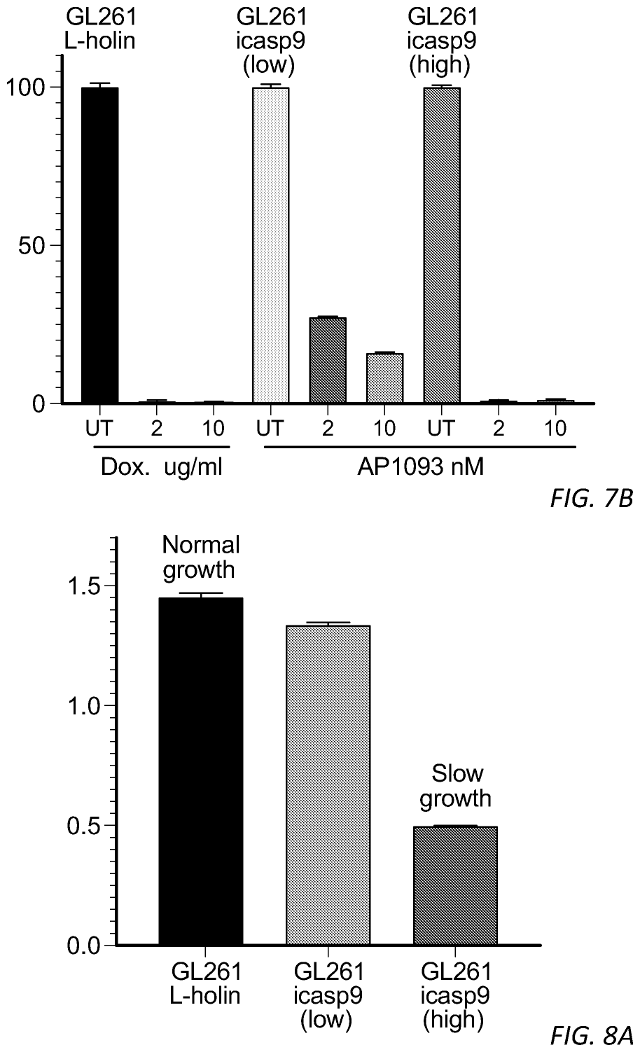

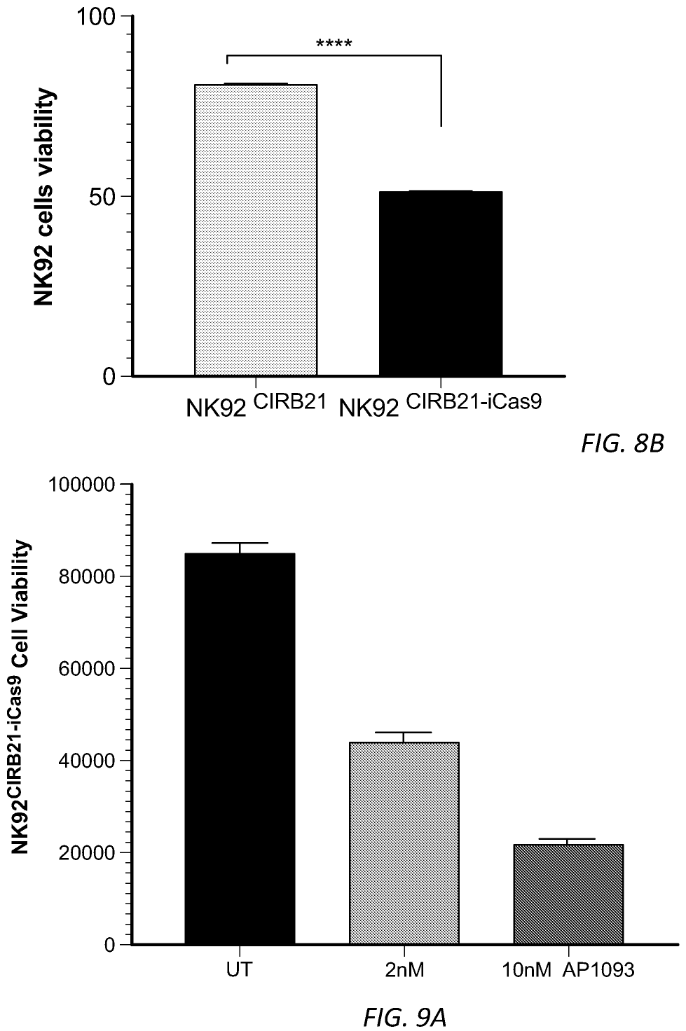

As shown in FIGS. 7A-7B, L-Holin expression was more effective at triggering cell death than icasp9 expressed at low levels. Similar cell death was observed for cells expressing L-Holin and cells expressing high levels of icasp9 (FIGS. 7A-7B). However, GL261 cells expressing high levels of icasp9 grew more slowly than GL261 expressing either low levels of icasp9 or L-Holin (FIG. 8A). Similar reduced growth rates were observed in NK92 cells expressing icasp9 (FIG. 8B).

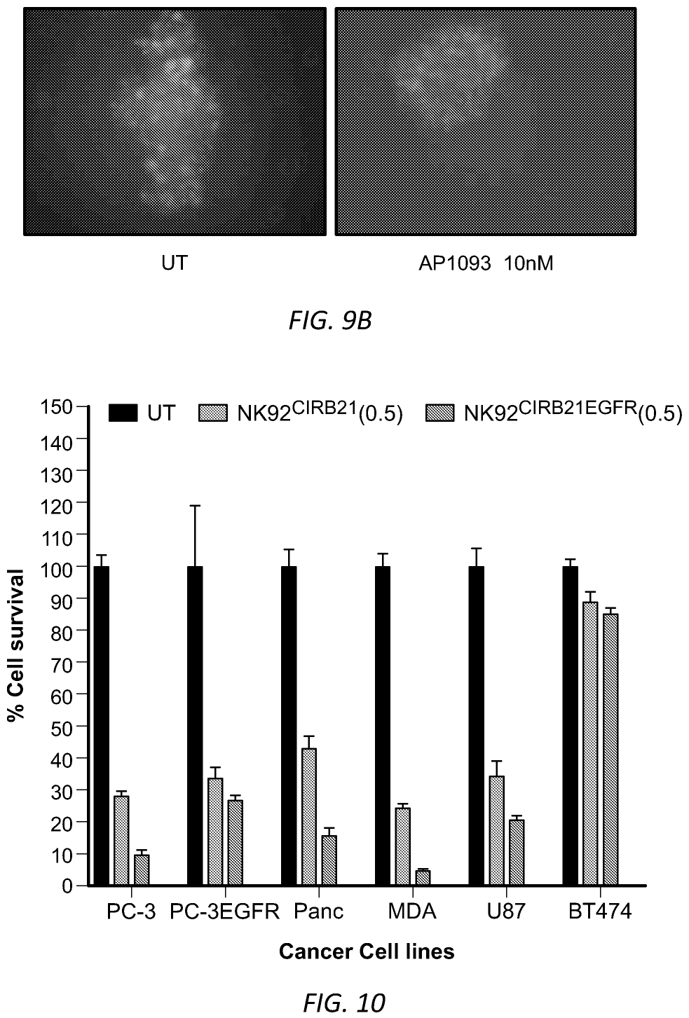

Next, we examined whether high doses of AP1093 could completely kill NK92^{CIRB21-icasp9} cells. As shown in FIG. 9A, NK92^{CIRB21-icasp9} cells remained viable in the presence of 10 nM AP1093. In NK92 cells expressing GFP and icasp9, NK92 cells were viable in 10 nM of AP1093 even after 5 days as measured GFP detection by immunofluorescence (FIG. 9B).

Taken together, these results demonstrate that expression of L-Holin provides improved cell killing with less negative effects on cell growth compared to expression of icasp9.

### Example 7. L-Holin Expressing Cells Remain Undetectable for at Least 2 Weeks Post Induction

Expression of L-Holin in NK92^{CIRB21+Holin} cells was induced with doxycycline. Total number of cells were counted after doxycycline treatment. Cells were washed to remove doxycycline and then cultured for 13 days. After 13 days of growth in the absence of doxycycline, total number of cells were counted. Untreated cells were counted as a control. As shown in FIG. 5B, L-Holin expressing cells remained undetectable even at 2 weeks post removal of doxycycline.

### Example 8. EGFR-CAR Improves the Cytotoxicity of NK92^{CIRB21} Cells

We compared cytotoxicity of NK92^{CIRB21EGFR} cells, which express EGFR-CAR, to NK92^{CIRB21}, which do not express the CAR, against multiple cancer cell lines. As shown in FIG. 10, NK92^{CIRB21} cells expressing EGFR-CAR showed enhanced killing of several cancer cell lines compared to parental NK92^{CIRB21} cells. These results demonstrate that NK92 cells expressing CIRB21 can synergize with other therapeutics such as EGFR-CAR.

### Example 9. NK92^{CIRB} Cells are More Sensitive to Lactate Dehydrogenase Inhibition Than NK92^{CIRB21} Cells

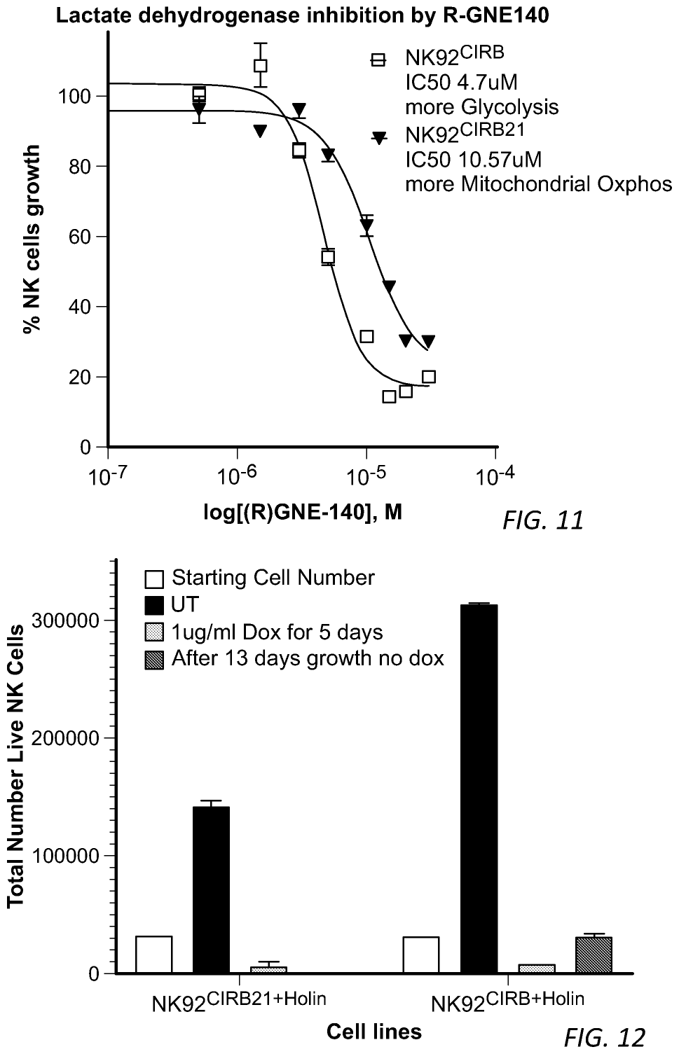

The ability of NK92^{CIRB} and NK92^{CIRB21} cells to grow in the presence of the lactate dehydrogenase inhibitor R-GNE140 was tested. Cells were incubated with increasing concentrations of R-GNE140 and total number of cells were counted. As shown in FIG. 11, the IC50 of R-GNE140 for inhibiting growth of NK92^{CIRB} cells and NK92^{CIRB21} cells was 4.7 μM and 10.57 μM, respectively. These results demonstrate that R-GNE140 inhibited growth of NK92^{CIRB} cells more than growth of NK92^{CIRB21} cells, suggesting that cell growth for NK92^{CIRB} is driven by glycolysis and cell growth for NK92^{CIRB21} is driven by mitochondria.

### Example 10. NK92^{CIRB21} Cells are More Sensitive to L-Holin Induced Cell Death Than NK92^{CIRB} Cells

The ability of L-holin to trigger death of NK92^{CIRB} cells and NK92^{CIRB21} cells was tested. L-bolin expression was induced in NK92^{CIRB} cells and NK92^{CIRB21} cells with doxycycline. Cells were washed to remove doxycycline and then cells were cultured for 13 days. Total number of cells were counted after 13 days of growth in the absence of doxycycline. Untreated cells were counted as a control. As shown in FIG. 12, NK92^{CIRB21} cells were undetectable after 13 days of growth in the absence of doxycycline while some NK92^{CIRB} cells survived and were detectable. These results demonstrate that NK92^{CIRB21} cells are more sensitive to killing by L-holin than NK92^{CIRB} cells. This sensitivity is consistent with the reliance of NK92^{CIRB21} cells on mitochondria, which are lysed by L-holin during cell killing.

### Example 11. Co-Incubation With Cancer Cells Induces Expression of Immune Checkpoint Proteins on the Surface of NK92^{CIRB21} Cells

NK92 cells expressing CIRB21 (NK92^{CIRB21} cells) were incubated with increasing numbers of prostate cancer cells PC-3 (N=300K/400K/500K). NK92 cells and PC-3 cells were co-incubated for 4 days. Following exposure, NK92^{CIRB21} cells were transferred to a fresh well and allowed to recover for 24 hours. Following recovery, NK92^{CIRB21} cells were analyzed by flow cytometry for immune checkpoint expression at the cell surface. Untreated NK92^{CIRB21} cells were used as a control. Experiments were performed without antibody as a negative control.

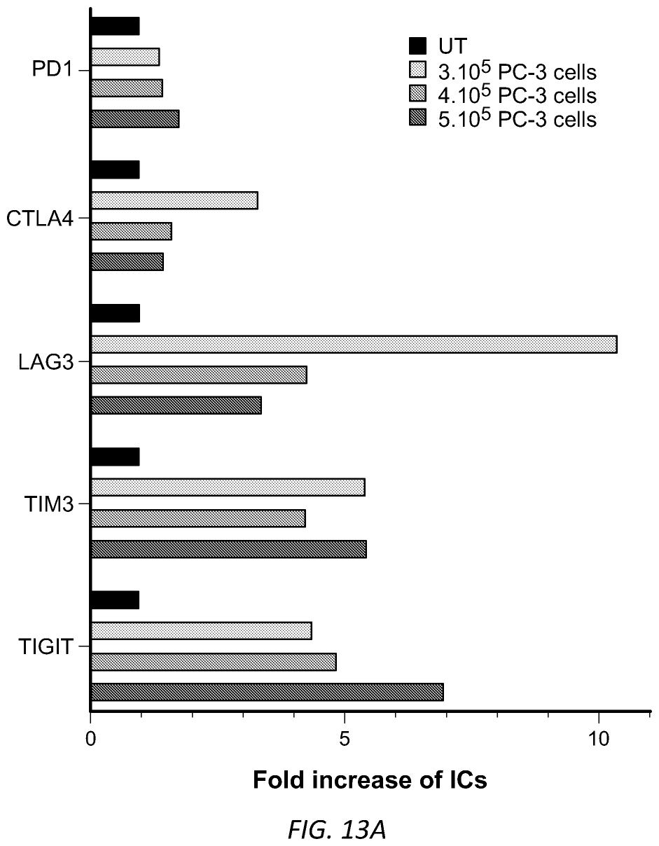

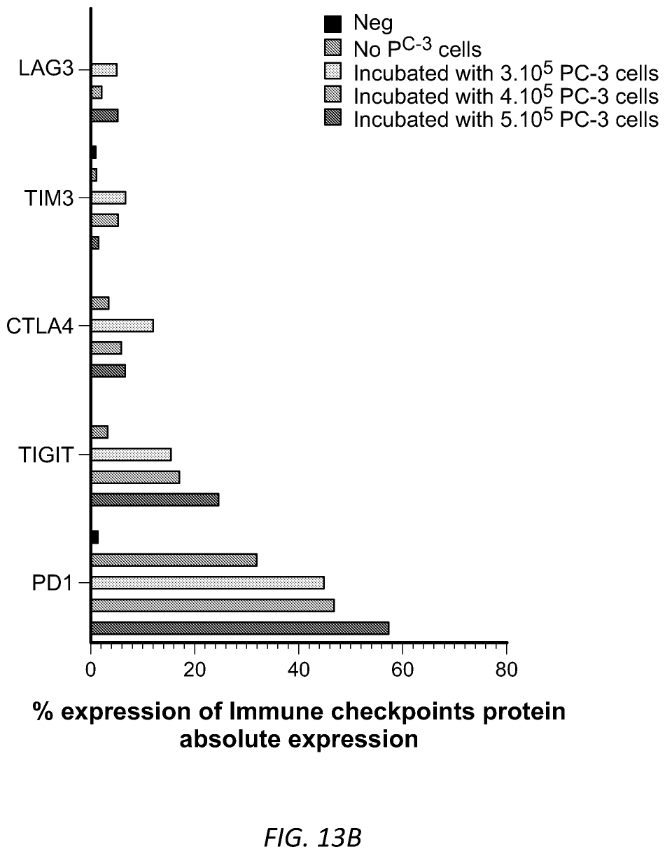

The percentage of NK92^{CIRB21} cells that express each immune checkpoint was determined by flow cytometry (Table 1). This data was used to determine the fold increase in expression of immune checkpoint proteins on NK92^{CIRB21} cells (FIG. 13A) and the absolute percent of NK92^{CIRB21} cells expressing immune checkpoint proteins (FIG. 13B).

Taken together, these results demonstrate that co-incubation of NK92^{CIRB21} cells with cancer cells induces expression of immune checkpoint proteins.

TABLE 1
Flow cytometry analysis of immune checkpoint
expression on NK92^{CIRB21} cells

| | NEG | UT | 300K | 400K | 500K |
|---|---|---|---|---|---|
| PD1 | 1.48% | 32.1% | 45% | 47.1% | 57.5% |
| CTLA4 | 0.77% | 3.69% | 12.3% | 6.05% | 5.49% |
| LAG3 | 0.52% | 0.52% | 5.41% | 2.24% | 1.77% |
| TIM3 | 1.15% | 1.27% | 6.91% | 5.42% | 6.94% |
| TIGIT | 0.79% | 3.55% | 15.6% | 17.3% | 24.8% |

### REFERENCES

1. Caligiuri M A. Human natural killer cells. Blood 2008; 112(3):461-9.
2. Vidal S M, Khakoo S I, Biron C A. Natural killer cell responses during viral infections: flexibility and conditioning of innate immunity by experience. Curr Opin Virol 2011; 1(6):497-512.
3. Orr M T, Lanier L L. Natural killer cell education and tolerance. Cell 2010; 142(6):847-56.
4. Malek T R. The biology of interleukin-2. Annu Rev Immunol 2008; 26:453-79.
5. Dahlberg C I, Sarhan D, Chrobok M, Duru A D, Alici E. Natural Killer Cell-Based Therapies Targeting Cancer: Possible Strategies to Gain and Sustain Anti-Tumor Activity. Front Immunol 2015; 6:605.
6. Maas R A, Dullens H F, Den Otter W. Interleukin-2 in cancer treatment: disappointing or (still) promising? A review. Cancer Immunol Immunother 1993; 36(3):141-8.
7. Glauser FL, DeBlois G, Bechard D, Fowler A A, Merchant R, Fairman R P. Cardiopulmonary toxicity of adoptive immunotherapy. Am J Med Sci 1988; 296(6):406-12.
8. Ardizzoni A, Bonavia M, Viale M, Baldini E, Mereu C, Verna A, et al. Biologic and clinical effects of continuous infusion interleukin-2 in patients with non-small cell lung cancer. Cancer 1994; 73(5):1353-60.
9. Donohue J H, Rosenberg S A. The fate of interleukin-2 after in vivo administration. J Immunol 1983; 130(5): 2203-8.
10. Shevach E M. Regulatory T cells in autoimmmunity*. Annu Rev Immunol 2000; 18:423-49.
11. Parkhurst M R, Riley J P, Dudley M E, Rosenberg S A. Adoptive transfer of autologous natural killer cells leads to high levels of circulating natural killer cells but does not mediate tumor regression. Clin Cancer Res 2011; 17(19):6287-97.
12. Rubnitz J E, Inaba H, Ribeiro R C, Pounds S, Rooney B, Bell T, et al. NKAML: a pilot study to determine the safety and feasibility of haploidentical natural killer cell transplantation in childhood acute myeloid leukemia. J Clin Oncol 2010; 28(6):955-9.
13. Vales-Gomez M, Reyburn H T, Mandelboim M, Strominger J L. Kinetics of interaction of HLA-C ligands with natural killer cell inhibitory receptors. Immunity 1998; 9(3):337-44.
14. Miller J S, Soignier Y, Panoskaltsis-Mortari A, MeNcarney S A, Yun G H, Fautsch S K, et al. Successful adoptive transfer and in vivo expansion of human haploidentical NK cells in patients with cancer. Blood 2005; 105(8):3051-7.
15. Geller M A, Cooley S, Judson P L, Ghebre R, Carson L F, Argenta P A, et al. A phase II study of allogeneic natural killer cell therapy to treat patients with recurrent ovarian and breast cancer. Cytotherapy 2011; 13(1):98-107.
16. Nagashima S, Mailliard R, Kashii Y. Reichert T E, Herberman R B, Robbins P, et al. Stable transduction of the interleukin-2 gene into human natural killer cell lines and their phenotypic and functional characterization in vitro and in vivo. Blood 1998; 91(10):3850-61.
17. Konstantinidis K V, Alici E, Aints A, Christensson B, Ljunggren H G, Dilber M S. Targeting IL-2 to the endoplasmic reticulum confines autocrine growth stimulation to NK-92 cells. Exp Hematol 2005; 33(2):159-64.
18. Mao Y, Sarhan D, Steven A, Seliger B, Kiessling R, Lundqvist A. Inhibition of tumor-derived prostaglandin-e2 blocks the induction of myeloid-derived suppressor cells and recovers natural killer cell activity. Clin Cancer Res 2014; 20(15):4096-106.
19. Bluestone J A, Abbas A K. Natural versus adaptive regulatory T cells. Nat Rev Immunol 2003; 3(3):253-7.
20. Rickert M, Wang X, Boulanger M J, Goriatcheva N, Garcia K C. The structure of interleukin-2 complexed with its alpha receptor. Science 2005; 308(5727):1477-80.
21. Leonard W J, Depper J M, Crabtree G R, Rudikoff S, Pumphrey J, Robb R J, et al. Molecular cloning and expression of cDNAs for the human interleukin-2 receptor. Nature 1984; 311(5987):626-31.
22. Hatakeyama M, Tsudo M, Minamoto S, Kono T, Doi T, Miyata T, et al. Interleukin-2 receptor beta chain gene: generation of three receptor forms by cloned human alpha and beta chain cDNA's. Science 1989; 244(4904):551-6.
23. Takeshita T, Asao H, Ohtani K, Ishii N, Kumaki S, Tanaka N, et al. Cloning of the gamma chain of the human IL-2 receptor. Science 1992; 257(5068):379-82.
24. Stauber D J, Debler E W, Horton P A, Smith K A, Wilson I A. Crystal structure of the IL-2 signaling complex: paradigm for a heterotrimeric cytokine receptor. Proc Natl Acad Sci U S A 2006; 103(8):2788-93.
25. Voss S D, Sondel P M, Robb R J. Characterization of the interleukin 2 receptors (IL-2R) expressed on human natural killer cells activated in vivo by IL-2: association of the p64 IL-2R gamma chain with the IL-2R beta chain in functional intermediate-affinity IL-2R. J Exp Med 1992; 176(2):531-41.
26. Caligiuri M A, Zmuidzinas A, Manley T J, Levine H, Smith K A, Ritz J. Functional consequences of interleukin 2 receptor expression on resting human lymphocytes. Identification of a novel natural killer cell subset with high affinity receptors. J Exp Med 1990; 171(5):1509-26.
27. Levin A M, Bates D L, Ring A M, Krieg C, Lin J T, Su L, et al. Exploiting a natural conformational switch to engineer an interleukin-2 ‘superkine’. Nature 2012; 484(7395):529-33.
28. Vihinen M. Relationship of protein flexibility to thermostability. Protein Eng 1987; 1(6):477-80.
29. Lohr M, Schmidt C, Ringel J, Kluth M, Muller P, Nizze H, et al. Transforming growth factor-betal induces desmoplasia in an experimental model of human pancreatic carcinoma. Cancer Res 2001; 61(2):550-5.
30. Ghiringhelli F, Menard C, Terme M, Flament C, Taieb J, Chaput N, et al. CD4+CD25+ regulatory T cells inhibit natural killer cell functions in a transforming growth factor-beta-dependent manner. J Exp Med 2005; 202(8):1075-85.
31. Li H, Han Y, Guo Q, Zhang M, Cao X. Cancer-expanded myeloid-derived suppressor cells induce anergy of NK cells through membrane-bound TGF-beta 1. J Immunol 2009; 182(1):240-9.
32. Clayton A, Mitchell J P, Court J, Linnane S, Mason M D, Tabi Z. Human tumor-derived exosomes down-modulate NKG2D expression. J Immunol 2008; 180(11):7249-58.
33. Clayton A, Mitchell J P, Court J, Mason M D, Tabi Z. Human tumor-derived exosomes selectively impair lymphocyte responses to interleukin-2. Cancer Res 2007; 67(15):7458-66.
34. Donatelli S S, Zhou J M, Gilvary D L, Eksioglu E A, Chen X, Cress W D, et al. TGF-beta-inducible microRNA-183 silences tumor-associated natural killer cells. Proc Natl Acad Sci U S A 2014; 111(11):4203-8.
35. Hsu A K, Quach H, Tai T, Prince H M, Harrison S J, Trapani J A, et al. The immunostimulatory effect of lenalidomide on NK-cell function is profoundly inhibited by concurrent dexamethasone therapy. Blood 2011; 117(5):1605-13.
36. Barnes P J. Anti-inflammatory actions of glucocorticoids: molecular mechanisms. Clin Sci (Lond) 1998; 94(6):557-72.
37. Boumpas D T, Anastassiou E D, Older S A, Tsokos G C, Nelson D L, Balow J E. Dexamethasone inhibits human interleukin 2 but not interleukin 2 receptor gene expression in vitro at the level of nuclear transcription. J Clin Invest 1991; 87(5):1739-47.
38. Tam Y K, Maki G, Miyagawa B, Hennemann B, Tonn T, Klingemann H G. Characterization of genetically altered, interleukin 2-independent natural killer cell lines suitable for adoptive cellular immunotherapy. Hum Gene Ther 1999; 10(8):1359-73.
39. Tai YT , Dillon M, Song W, Leiba M, Li X F, Burger P, et al. Anti-CSI humanized monoclonal antibody HuLuc63 inhibits myeloma cell adhesion and induces antibody-dependent cellular cytotoxicity in the bone marrow milieu. Blood 2008; 112(4):1329-37.
40. Glienke W, Esser R, Priesner C, Suerth J D, Schambach A, Wels W S, et al. Advantages and applications of CAR-expressing natural killer cells. Front Pharmacol 2015; 6:21.
41. Tonn T, Schwabe D, Klingemann HG, Becker S, Esser R. Kochl U, et al. Treatment of patients with advanced cancer with the natural killer cell line NK-92. Cytotherapy 2013; 15(12):1563-70.
42. Arai S, Meagher R, Swearingen M, Myint H, Rich E, Martinson J, et al. Infusion of the allogeneic cell line NK-92 in patients with advanced renal cell cancer or melanoma: a phase I trial. Cytotherapy 2008; 10(6):625-32.
43. Gong J H, Maki G, Klingemann H G. Characterization of a human cell line (NK-92) with phenotypical and functional characteristics of activated natural killer cells. Leukemia 1994; 8(4):652-8.
44. Suerth J D, Schambach A, Baum C. Genetic modification of lymphocytes by retrovirus-based vectors. Curr Opin Immunol 2012; 24(5):598-608.
45. Terme M, Ullrich E, Delahaye N F, Chaput N, Zitvogel L. Natural killer cell-directed therapies: moving from unexpected results to successful strategies. Nat Immunol 2008; 9(5):486-94.
46. Yang X O, Nurieva R, Martinez G J, Kang H S, Chung Y, Pappu B P, et al. Molecular antagonism and plasticity of regulatory and inflammatory T cell programs. Immunity 2008; 29(1):44-56.
47. Vihinen M, Torkkila E, Riikonen P. Accuracy of protein flexibility predictions. Proteins 1994; 19(2):141-9.
48. Nagler A, Lanier L L, Phillips J H. The effects of IL-4 on human natural killer cells. A potent regulator of IL-2 activation and proliferation. J Immunol 1988; 141(7):2349-51.
49. Ke L D, Shi Y X, Yung W K. VEGF(121), VEGF(165) overexpression enhances tumorigenicity in U251 MG but not in NG-1 glioma cells. Cancer Res 2002; 62(6):1854-61.
50. Kaighn M E, Narayan K S, Ohnuki Y, Lechner J F, Jones L W. Establishment and characterization of a human prostatic carcinoma cell line (PC-3). Invest Urol 1979; 17(1):16-23.
51. Fujii H, Nakagawa Y, Schindler U, Kawahara A, Mori H, Gouilleux F, et al. Activation of Stats by interleukin 2 requires a carboxyl-terminal region of the interleukin 2 receptor beta chain but is not essential for the proliferative signal transmission. Proc Natl Acad Sci U S A 1995; 92(12):5482-6.
52. Asao H, Okuyama C, Kumaki S, Ishii N, Tsuchiya S, Foster D, et al. Cutting edge: the common gamma-chain is an indispensable subunit of the IL-21 receptor complex. J Immunol 2001; 167(1):1-5.
53. Strengell M, Sareneva T, Foster D, Julkunen I, Matikainen S. IL-21 up-regulates the expression of genes associated with innate immunity and Th1 response. J Immunol 2002; 169(7):3600-5.
54. Zeng R, Spolski R, Casas E, Zhu W, Levy D E, Leonard W J. The molecular basis of IL-21-mediated proliferation. Blood 2007; 109(10):4135-42.
55. Strengell M, Matikainen S, Siren J, Lehtonen A, Foster D, Julkunen I, et al. IL-21 in synergy with IL-15 or IL-18 enhances IFN-gamma production in human NK and T cells. J Immunol 2003; 170(11):5464-9.
56. Spolski R, Leonard W J. Interleukin-21: a double-edged sword with therapeutic potential. Nat Rev Drug Discov 2014; 13(5):379-95.
57. Endo T A, Masuhara M, Yokouchi M, Suzuki R, Sakamoto H, Mitsui K, et al. A new protein containing an SH2 domain that inhibits JAK kinases. Nature 1997; 387(6636):921-4.
58. Cohney S J, Sanden D, Cacalano N A, Yoshimura A, Mui A, Migone T S, et al. SOCS-3 is tyrosine phosphorylated in response to interleukin-2 and suppresses STAT5 phosphorylation and lymphocyte proliferation. Mol Cell Biol 1999; 19(7):4980-8.
59. Bromberg J F, Wrzeszczynska M H, Devgan G, Zhao Y, Pestell R G, Albanese C, et al. Stat3 as an oncogene. Cell 1999; 98(3):295-303.
60. Bromberg J. Stat proteins and oncogenesis. J Clin Invest 2002; 109(9): 1139-42.
61. Chin Y E, Kitagawa M, Su W C, You Z H, Iwamoto Y, Fu X Y. Cell growth arrest and induction of cyclin-dependent kinase inhibitor p21 WAF1/CIP1 mediated by STAT1. Science 1996; 272(5262):719-22.
62. Kaplan D H, Shankaran V, Dighe A S, Stockert E, Aguet M, Old L J, et al. Demonstration of an interferon gamma-dependent tumor surveillance system in immunocompetent mice. Proc Natl Acad Sci U S A 1998; 95(13):7556-61.
63. Qing Y, Stark G R. Alternative activation of STAT1 and STAT3 in response to interferon-gamma. J Biol Chem 2004; 279(40):41679-85.
64. Parrish-Novak J, Dillon S R, Nelson A, Hammond A, Sprecher C, Gross J A, et al. Interleukin 21 and its receptor are involved in NK cell expansion and regulation of lymphocyte function. Nature 2000; 408(6808):57-63.

### OTHER EMBODIMENTS

It is to be understood that while the invention has been described in conjunction with the detailed description thereof, the foregoing description is intended to illustrate and not limit the scope of the invention, which is defined by the scope of the appended claims. For example, although human cells and sequences are exemplified herein, e.g., for use in treating human subjects, sequences and NK cells from other species can also be used. Other aspects, advantages, and modifications are within the scope of the following clams.

What is claimed is:

1. A nucleic acid or a set of nucleic acids encoding a fusion protein and a pore-forming protein, wherein the fusion protein comprises interleukin 2 (IL2) fused to the N-terminus of interleukin 2 receptor beta (IL2Rβ), with an intervening linker.

2. The nucleic acid or the set of nucleic acids of claim 1, wherein:
the IL2 comprises SEQ ID NO: 34, and/or
the IL2Rβ comprises amino acids 27-551 of SEQ ID NO: 35.

3. The nucleic acid or the set of nucleic acids of claim 1, wherein the intervening linker between IL2 and the N-terminus of IL2Rβ comprises an extracellular domain of IL2Rα.

4. The nucleic acid or the set of nucleic acids of claim 3, wherein the extracellular domain of IL2Rα comprises SEQ ID NO: 28.

5. The nucleic acid or the set of nucleic acids of claim 1, further comprising a cytoplasmic domain of IL21R at the C-terminus of IL2Rβ, optionally with an intervening linker therebetween.

6. The nucleic acid or the set of nucleic acids of claim 5, wherein the cytoplasmic domain of IL21R comprises amino acids 254-538 of SEQ ID NO: 36.

7. The nucleic acid or the set of nucleic acids of claim 1, further comprising an activation domain of CD28 at the C-terminus of the IL2Rβ portion, optionally with an intervening linker therebetween.

8. The nucleic acid or the set of nucleic acids of claim 7, wherein the activation domain of CD28 comprises amino acids 180 to 220 of SEQ ID NO: 38.

9. The nucleic acid or the set of nucleic acids of claim 1, wherein the pore-forming protein is a holin.

10. The nucleic acid or the set of nucleic acids of claim 9, wherein the holin is lambda holin.

11. The nucleic acid or the set of nucleic acids of claim 10, wherein the lambda holin comprises SEQ ID NO: 1.

12. The nucleic acid or the set of nucleic acids of claim 1, further comprising one or more regulatory regions for expression of the fusion protein, the pore-forming protein, or both.

13. The nucleic acid or the set of nucleic acids of claim 12, wherein the one or more regulatory regions comprises one or more inducible promoters.

14. The nucleic acid or the set of nucleic acids of claim 13, the one or more inducible promoters is a tetracycline-inducible promoter, a steroid-inducible promoter, an interferon-inducible promoter, a cumate-inducible promoter, a heavy metal-inducible promoter, or a combination thereof.

15. The nucleic acid or the set of nucleic acids of claim 1, wherein the nucleic acid or the set of nucleic acids encoding the fusion protein and the pore-forming protein is comprised in a vector.

16. The nucleic acid or the set of nucleic acids of claim 15, wherein the vector is a viral vector.

17. The nucleic acid or the set of nucleic acids of claim 16, wherein the viral vector is a lentiviral vector, a retroviral vector, an adenoviral vector, or an adeno-associated viral vector.

18. An immune cell comprising the nucleic acid or the set of nucleic acids of claim 1.

19. The immune cell of claim 18, further comprising a nucleic acid encoding CD16, NKP44, NKP46, NKP30, or a combination thereof.

20. The immune cell of claim 18, wherein the immune cell is a natural killer (NK) cell or a T cell.

21. A method of treating a subject having cancer, the method comprising administering a therapeutically effective amount of the immune cell of claim 18 to a subject in need thereof.

22. The method of claim 21, wherein the subject has a solid tumor.

23. The method of claim 21, further comprising administering one or more of an anti-tumor monoclonal antibody or a checkpoint inhibitor.

24. The method of claim 23, wherein the checkpoint inhibitor is selected from the group consisting of an anti-CTLA-4 antibody, an anti-PD-1 antibody, an anti-PD-L1 antibody, an anti-CD4-antibody, an anti-Tim3 antibody, an anti-Lag3 antibody, an anti-TIGIT antibody, and a combination thereof.

25. The method of claim 21, wherein the immune cell is administered intravenously.

26. The method of claim 21, wherein the immune cell is subjected to 500 to 1000 cGy of gamma irradiation prior to being administered.

27. The method of claim 21, wherein the immune cell is a natural killer (NK) cell.

28. A method of treating a subject having graft-versus-host disease (GVHD) or an autoimmune disease, the method comprising administering a therapeutically effective amount of the immune cell of claim 18 to a subject in need thereof.

29. The method of claim 28, wherein the immune cell is administered intravenously.

30. The method of claim 28, wherein the immune cell is subjected to 500 to 1000 cGy of gamma irradiation prior to being administered.

31. The method of claim 28, wherein the immune cell is a regulatory T (T-reg) cell.


SEQUENCE LISTING (39 sequences)

SEQ ID NO: 1 - protein, 105 aa, Unknown
  Enterobacteriaceae
MPEKHDLLAAILAAKEQGIGAILAFAMAYLRGRYNGGAFTKTVIDATMCAIIAWFIRDLLDFAGLSSNLAYITSVFIGYIGTDSIGSLIKRFAAKKAGVEDGRNQ

SEQ ID NO: 2 - DNA, 31 bp, Artificial
  Primer
tgcaggatccactcacagtaacctcaactcc

SEQ ID NO: 3 - DNA, 30 bp, Artificial
  Primer
tgcactcgagagtgaaaccattttagagcc

SEQ ID NO: 4 - DNA, 94 bp, Artificial
  Primer
ggattaccttttgtcaaagcatcatctcaacactgactgagcagaagctcatttcggaagaagaccttgaaatggagaccagtcagtttccagg

SEQ ID NO: 5 - DNA, 65 bp, Artificial
  Primer
cctgatatgttttaagtgggaagcacttaattatcagattgttcttctactcttcctctgtctcc

SEQ ID NO: 6 - DNA, 31 bp, Artificial; identical to SEQ ID NO: 2
  Primer

SEQ ID NO: 7 - DNA, 41 bp, Artificial
  Primer
gggaagtgccattcaccgcgcaggaagtctcactctcagga

SEQ ID NO: 8 - DNA, 48 bp, Artificial
  Primer
ggctctcgagttgtagaagcatgtgaactgggaagtgccattcaccgc

SEQ ID NO: 9 - DNA, 27 bp, Artificial
  Primer
catcttcagtgcctagaagaagaactc

SEQ ID NO: 10 - DNA, 27 bp, Artificial
  Primer
gagttcttcttctaggcactgaagatg

SEQ ID NO: 11 - DNA, 45 bp, Artificial
  Primer
ttcccagttcacatgcttctacaagtcgacagccaacatctcctg

SEQ ID NO: 12 - DNA, 50 bp, Artificial
  Primer
agcttctagactcgagttatcacaccaagtgagttgggtcctgaccctgg

SEQ ID NO: 13 - DNA, 318 bp, Artificial
  optimized version of L-Holin for improved expression in mammals
atgccggaaaaacacgatctgctggcggcgattctggcggcgaaggaacagggtattggcgcgattctggcgtttgcgatggcgtacctgcgtggtcgttataacggtggcgcgttcaccaagaccgtgatcgacgcgaccatgtgcgcgatcattgcgtggttcattcgtgacctgctggattttgcgggtctgagcagcaacctggcgtacatcaccagcgtttttatcggttatattggcaccgatagcattggcagcctgattaagcgttttgcggcgaagaaagcgggtgtggaagacggtcgtaaccaataa

SEQ ID NO: 14 - DNA, 39 bp, Artificial
  Primer
gagaataccggttgcgctgccaccatgccggaaaaacac

SEQ ID NO: 15 - DNA, 28 bp, Artificial
  Primer
agacggtcgtaaccaaggatccggagag

SEQ ID NO: 16 - DNA, 29 bp, Artificial
  expression cassette
aattcactttggccgcgaatcgatatgtc

SEQ ID NO: 17 - DNA, 341 bp, Artificial
  expression cassette - TRE promoter
gagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcctggagaataccggttgcgctgccacc

SEQ ID NO: 18 - DNA, 324 bp, Artificial
  expression cassette - L-Holin
atgccggaaaaacacgatctgctggcggcgattctggcggcgaaggaacagggtattggcgcgattctggcgtttgcgatggcgtacctgcgtggtcgttataacggtggcgcgttcaccaagaccgtgatcgacgcgaccatgtgcgcgatcattgcgtggttcattcgtgacctgctggattttgcgggtctgagcagcaacctggcgtacatcaccagcgtttttatcggttatattggcaccgatagcattggcagcctgattaagcgttttgcggcgaagaaagcgggtgtggaagacggtcgtaaccaaggatccgga

SEQ ID NO: 19 - DNA, 54 bp, Artificial
  expression cassette - T2A
gagggcagaggaagtctgctaacatgcggtgacgtcgaggagaatcctggccca

SEQ ID NO: 20 - DNA, 717 bp, Artificial
  expression cassette - GFP
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 21 - DNA, 10 bp, Artificial
  expression cassette - NheI site
gctagcatcc

SEQ ID NO: 22 - DNA, 233 bp, Artificial
  expression cassette - EF-1
gggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagactgcgatcgcaatgtacagt

SEQ ID NO: 23 - DNA, 53 bp, Artificial
  expression cassette - Puro
atgaccgagtacaagcccacggtgcgcctcgccacccgcgacgacgtccccag

SEQ ID NO: 24 - DNA, 546 bp, Artificial
  expression cassette - Puro
cgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggttccggc

SEQ ID NO: 25 - DNA, 57 bp, Artificial
  expression cassette - P2A
gcaacaaacttctctctgctgaaacaagccggagatgtcgaagagaatcctggaccg

SEQ ID NO: 26 - DNA, 747 bp, Artificial
  expression cassette - rTTA
atgtctagactggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaa

SEQ ID NO: 27 - skipped

SEQ ID NO: 28 - protein, 27 aa, human
EMETSQFPGEEKPQASPEGRPESETSC

SEQ ID NO: 29 - protein, 10 aa, human
EQKLISEEDL

SEQ ID NO: 30-33 - skipped (4 entries)

SEQ ID NO: 34 - protein, 153 aa, human
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 35 - protein, 551 aa, human
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 36 - protein, 538 aa, human
MPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 37 - skipped

SEQ ID NO: 38 - protein, 220 aa, human
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 39 - protein, 41 aa, human
RSKRSRLLHSDYMNMTPRRPGPTRKHYQPYAPPRDFAAYRS